(19) United States
(12) Patent Application Publication
(10) Pub. No.: US 2003/0129591 A1
(43) Pub. Date: Jul. 10, 2003

(54) Linker arms for nanocrystals and compounds thereof

(76) Inventors: Sandra J. Rosenthall, Nashville, TN (US); Ian D. Tomlinson, Nashville, TN (US)

Correspondence Address: STITES & HARBISON PLLC, 424 CHURCH STREET, SUITE 1800, NASHVILLE, TN, 37219-2376, US

(21) Appl. No.: 09/864,731

(22) Filed: May 24, 2001

Related U.S. Application Data
(60) Provisional application No. 60/206,771, filed on May 24, 2000.

(51) Int. Cl. C12Q 1/68; C12Q 1/00; G01N 33/53; C07F 3/08; C07F 7/24

(52) U.S. Cl. 435/6; 435/7.1; 546/2; 548/402; 544/225

(57) ABSTRACT

Nanocrystal compounds and nanocrystal compound linker arm of the following formula:

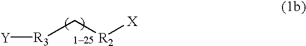

wherein Y is the attachment point for a nanocrystal, X is an attachment point of an organic compound. R₂ is a bond or selected from the group consisting of carbonyl, O, NH, S, CONH, COO, S, C₁₋₁₀ alkyl, carbamate, and thiocarbamate. R₃ is selected from the group consisting of: SH, O(CH₂₍ₙ₎O)ₙSH, NH(CH₂₍ₙ₎O)ₙSH, NH(CH₂₍ₙ₎NH)SH, S(CH₂₍ₙ₎O)ₙSH, S(CH₂₍ₙ₎S)SH, and a polyether chain. n is 1-10. S is attached to the nanocrystal.

### PRIORITY

This application claims priority under 35 U.S.C. §120 to Application No. 60/206,771, filed May 24, 2000, the contents of which are incorporated herein by reference.

### FIELD OF THE INVENTION

This invention generally relates to nanocrystals, linker arms for nanocrystals, and compounds resulting therefrom. Furthermore, this invention relates to labeling techniques using the compounds of the present invention.

### BACKGROUND OF THE INVENTION

Semi-conducting nanocrystals, also referred to as quantum dots, have many advantages over traditional dye molecules in the areas of fluorescent labeling. Fluorescent nanocrystal labeling has broad application in the biomedical sciences. For example, the labeling technique of the present invention provides improved and widely applicable methods for detecting biomolecules and for scrutinizing biomolecular processes.

Currently quantum dots are being used as fluorescent tags capable of tracing specific substances within cells. Quantum dots can be activated to glow with different colors, so it is easier to use quantum dots in tandem than combinations of conventional fluorescent dyes. See “Semiconductor Beacons Light up Cell Structures” Service, Science, Vol. 281. The conventional fluorescent dye, typically made from small organic dye molecules can be toxic, can quench quickly, and can be difficult to use in tandem, since typically each dye must be excited with photons at a different wavelength. Additionally, compared with conventional coloring agents such as rhodamine 6G or other organic dyes, the quantum dots produce narrower and much brighter fluorescence spectra. See “Quantum Dots Meet Biomolecules”, Jacoby. With the quantum dots, or nanocrystals, the absorbency onset and emission maxima shift to a higher energy with decreasing size. The excitation typically tracks the absorbency, resulting in a tunable fluorophore that can be excited efficiently at any wavelength shorter than the emission peak, yet will emit with the same characteristic a narrow, symmetric spectrum regardless of the excitation wavelength. See “Semiconductor Nanocrystals as Fluorescent Biological Labels”, Bruchez, et al., Science, Vol. 281, 1998. The absorbance onset and emission maximum shift to higher energy as the size of the nanocrystal decreases. Because the excitation tracks absorbance, the nanocrystals can be excited at many wavelengths, yet still they emit the same narrow, symmetric peak. By varying the material used or the size of the quantum dot, the color can be changed. Additionally, a range of quantum dots of different colors may be excited with a single wavelength and detected simultaneously. See “Bright Lights for Biomolecules”, Analytical Chemistry News and Features, December 1998. Thus, the quantum dots, or semiconducting nanocrystals, are much more flexible and advantageous when used in assays.

The attachment of biologically active ligands to nanocrystals including, for example, cadmium selenide nanocrystals, is a new method of producing novel fluorescent sensors. The sensors can have a variety of applications. They may be used in fundamental studies ranging from assay systems to locate the distribution and localization of membrane bound receptors, transporter proteins and channels in whole assay systems. They may also be used in novel methodologies for the development of pharmaceutically active compounds using high throughput screening.

The small size of the of the nanocrystal ligand conjugate offers advantages over conventional techniques that use antibodies bound to fluorescent dyes. These advantages include the small size of the drug nanocrystal conjugate, which enables it to fit into the synaptic gap. Antibody-fluorescent dye systems are much larger than the nanocrystal drug conjugates of the present invention, so the antibody-fluorescent dye stems are less likely to fit into the synaptic gap. Additionally most antibodies are cell permeable.

The increased photostability of the nanocrystals means that they are not as easily photo-bleached as conventional dyes. Therefore, the nanocrystal compounds of the present invention may be used in experiments that require longer periods of illumination without photo-bleaching becoming a major problem.

The increased brightness of the nanocrystals enhances the sensitivity of the assay systems when compared to traditional dyes. Therefore, assay systems can be developed that detect lower concentrations of the analyte.

Also see “Quantum Dot Bioconjugates for Ultrasensitive Nonisotopic Detection”, Chan, Nie, Science, Vol. 281, 1998.

There are several patents that disclose nanocrystals that can be used in connection with the present invention.

U.S. Pat. No. 5,990,479 to Weiss et al. discloses a luminescent nanocrystal compound that is capable of linking to an affinity molecule. Weiss et al. further describe a process for making luminescent semiconductor nanocrystal compounds and for making an organo luminescent semiconductor probe comprising the nanocrystal compound linked to an affinity molecule capable of bonding to a detectable substance and a process for using the probe to determine the presence of a detectable substance in a material.

U.S. Pat. No. 5,751,018 to Alivisatos et al. discloses methods for attaching semiconductor nanocrystals to solid inorganic surfaces, using self-assembled bifunctional organic monolayers as bridge compounds.

U.S. Pat. No. 5,537,000 to Alivisatos et al., which describes electroluminescent devices formed using semiconductor nanocrystals as an electron transport media and a method for making such electroluminescent devices.

U.S. Pat. No. 5,505,928 to Alivisatos et al. discloses nanocrystals of III-V semiconductors, and U.S. Pat. No. 5,262,352 Alivisatos et al. discloses a process for forming a solid, continuous thin film of a semiconductor material on a solid support surface.

### SUMMARY OF THE INVENTION

An embodiment of the present invention is to provide linker arms to attach organic compounds to nanocrystals, or quantum dots. A linker arm of the present invention may have the following formula:

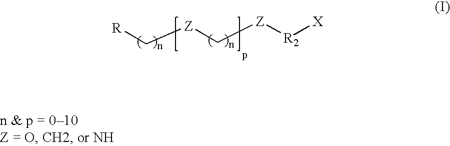

wherein Y represents the attachment point to the nanocrystal and X represents the attachment point of an organic compound.

R is a bond or is selected from the group consisting of:

SH,

O(CH₂₍ₙ₎O)ₙSH,

NH(CH₂₍ₙ₎O)ₙSH,

NH(CH₂₍ₙ₎NH)SH,

S(CH₂₍ₙ₎O)ₙSH, and

S(CH₂₍ₙ₎S)SH. n is 1-10, with S being attached to the nanocrystal.

R₂ is a bond or selected from the group consisting of carbonyl, NH, S, CONH, COO, S, C₁₋₁₀ alkyl, carbamate, and thiocarbamate.

When n and p are 1 or more, the resulting carbon or carbon chain may be substituted.

Preferably, z is CH₂. Preferably n and p are 1-5.

In another embodiment of the present invention, the linker arm may have the following formula:

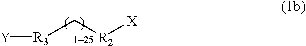

Wherein Y is the attachment point for a nanocrystal, X is an attachment point of an organic compound.

R₂ is a bond or selected from the group consisting of

carbonyl,

O,

NH,

S,

CONH,

COO,

S,

C₁₋₁₀ alkyl,

carbamate, and

thiocarbamate.

R₃ is selected from the group consisting of:

SH,

O(CH₂₍ₙ₎O)ₙSH

NH(CH₂₍ₙ₎O)ₙSH,

NH(CH₂₍ₙ₎NH)SH,

S(CH₂₍ₙ₎O)ₙSH,

S(CH₂₍ₙ₎S)SH, and

a polyether chain.

n is 1-10. S is attached to the nanocrystal.

Preferably, the organic compound is a biologically active compound. Examples of the biologically active compounds of the present invention include seratonin or seratonin derivatives, cocaine analogues, phenyl tropane analogues, phenylisopropylamine derivatives, dopamine derivatives, melatonin derivatives, chlormethiazole derivatives, derivatives of RTI-4229-75, and derivatives of GBR 12935. RTI-4229-75 and GBR 12935 are further described below.

For the purposes of providing examples only, the preferred organic compounds attached to the nanocrystal of the present invention specifically include the following:

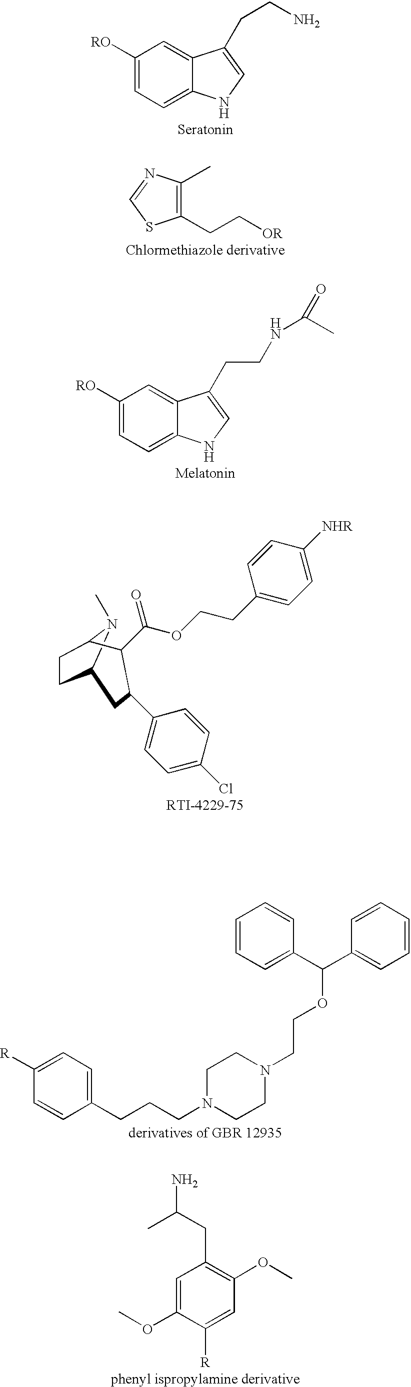

In the above examples, R represents the attachment point to the linker arm. Additionally, the R group may be “floating” when attached to the phenyl ring. That is, the R group may be attached to any available carbon atom on the ring.

The present invention further is directed to nanocrystal compounds, which include linker arm derivatives of the present invention. More specifically, the nanocrystal compounds of the present invention comprise a semiconducting nanocrystal and a linking arm having a first portion linked to the nanocrystal and a second portion linked to an organic compound.

Examples of nanocrystal compounds of the present invention include the following formulae (II), (III), (IV), (V), (VI), (VII), (X) and (XI):

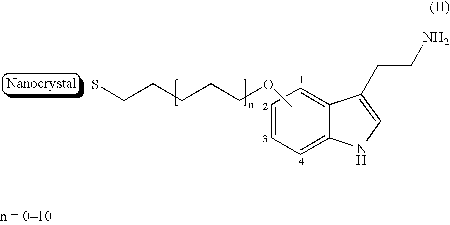

Preferably n is 2,3,4 or 5. The linker arm may be attached to positions 1,2,3, or 4. Most preferably, the linker arm is attached to position 2.

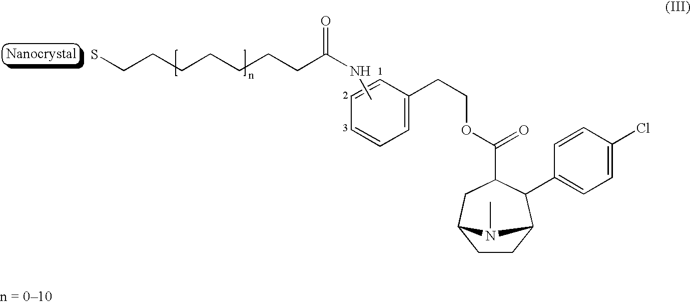

Preferably n is 1, 2, 3, or 4 and the linker arm is attached to positions 1, 2, 3, or 4. Most preferably, positions 1, 2, or 3. Most preferably, position 2.

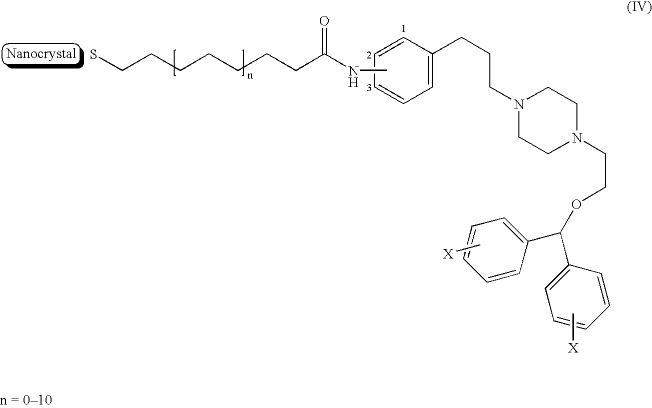

Preferably n is 1, 2, 3, 4 or 5 and the linker arm is attached to positions 1, 2, 3, or 4. Preferably, the linker arm is attached to one of positions 1, 2, or 3. Most preferably, position 3.

X═H or halogen. Preferably, X is H or F.

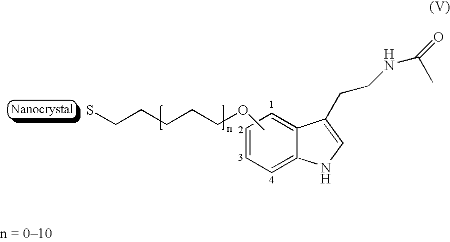

Preferably n is 2, 3, 4 or S. The linker arm may be attached to positions 1, 2, 3, or 4. Preferably, position 2.

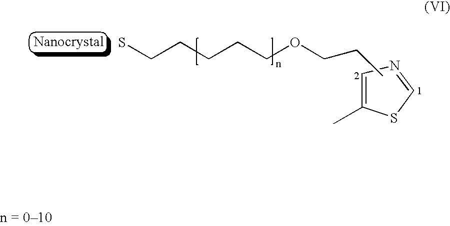

Preferably n is 2, 3, 4, or 5. The linker arm may be attached to positions 1 or 2. Preferably, position 2.

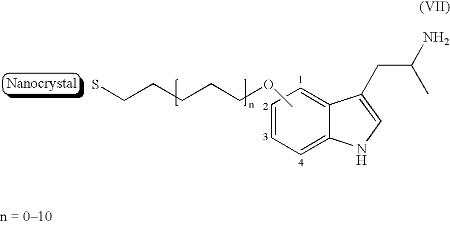

Preferably n is 2, 3, 4 or 5. The linker arm may be attached to positions 1,2,3,or 4. Preferably, position 2.

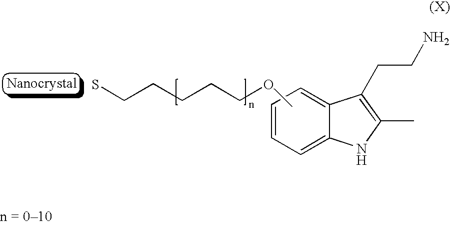

Preferably n is 2, 3, 4 or 5. The linker arm may be attached to positions 1, 2, 3, or 4. Preferably, position 2.

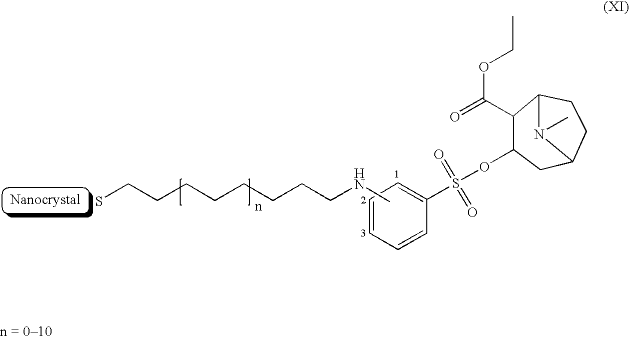

Preferably n is 2,3,4 or 5. The linker arm may be attached to positions 1,2,3,or 4. Preferably, position 2.

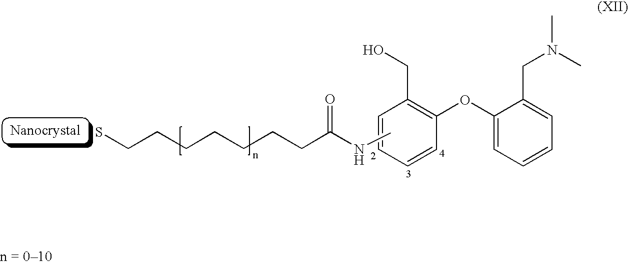

Preferably n is 2, 3, 4 or 5. The linker arm may be attached to positions 1, 2, 3, or 4. Preferably, position 2.

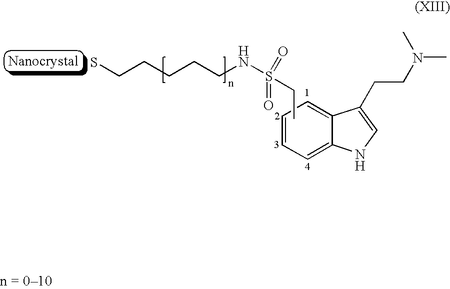

Preferably n is 2,3,4 or 5. The linker arm may be attached to positions 1, 2, 3, or 4. Preferably, position 2.

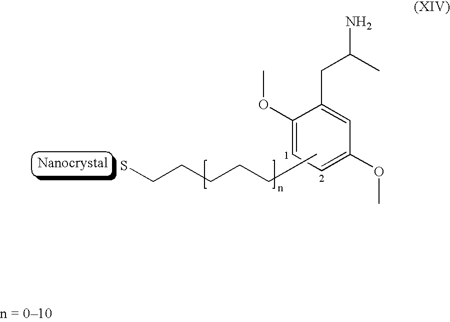

Preferably n is 2,3,4 or 5. The linker arm may be attached to positions 1 or 2. Preferably, position 2.

The linker arm attaching the compounds to the nanocrystal can be altered by attaching a polyethylene glycol to it. Additionally, the linker arm may be altered by replacing a carbon with an oxygen, sulfur, or NH group. The length of the linker arm may be increased or decreased and it may comprise chains with lengths of, for example, 1 to 10 carbons.

### DETAILED DESCRIPTION OF THE INVENTION

As stated above, the present invention relates to linker arms to which biologically active molecules can be attached to nanocrystals. The nanocrystals used in conjunction with the present invention are the nanocrystals typically used in fluorescent imaging techniques. Preferably, the nanocrystals used in conjunction with the present invention are semiconductor nanocrystals capable of luminescence and/or scattering or diffraction when excited by an electromagnetic radiation source (of broad or narrow bandwidth) or a particle beam, and capable of exhibiting a detectable change of absorption and/or emitting radiation in a narrow wavelength band and/or scattering or diffracting when excited. For exemplary purposes, the nanocrystals of U.S. Pat. No. 5,990,479 may be used with the present invention.

That is, in embodiments of the present invention, an organic or inorganic single crystal particle having an average cross-section of about 20 nanometers (nm) or 20×10⁻⁹ meters (200 Angstroms), preferably no larger than about 1 nm (100 Angstroms) and a minimum average cross-section of about 1 nm, although in some instances a smaller average cross-section nanocrystal, i.e., down to about 0.5 nm (5 Angstroms), may be acceptable. Typically the nanocrystal will have an average cross-section ranging in size from about 1 nm (10 Angstroms) to about 10 nm (100 Angstroms).

Furthermore, for exemplary purposes only, these nanocrystals include, but not are limited to CdSe, CdS, PbSe, PbS, and CdTe.

As mentioned above, there are disadvantages to traditional dye molecules that are used in the area of fluorescent labeling. For example, simultaneous localization of several different proteins in situ is currently limited by the wide emission spectra and photostabilities of fluorescent dyes traditionally used to study cell surface receptors, ion channels, and transporters. The nanocrystal compounds of the present invention can overcome the above deficiencies. For example, in one embodiment of the present invention, the nanocrystal compounds comprise core (CdSe)/shell(ZnS) semiconducting nanocrystals. Through quantum confinement, the fluorescent wavelength of these nanocrystals are continuously tunable by size. For example a 25 Angstrom nanocrystal of this embodiment emits at 455 nm while a 60 Angstrom nanocrystal of this embodiment emits at 625 nm. Unlike dye molecules and variants of green fluorescent protein, these nanocrystals have narrow gaussian emission spectra enabling multiplex imaging. The absorption of these nanocrystals is continuous above the band-gap; hence all sizes of nanocrystals can be excited with a single excitation wavelength. In addition, the nanocrystals of this embodiment are much brighter than traditional dyes, even hours after continuous illumination.

The present invention further relates to multiple organic compounds in combination with the linker arms of the present invention. The present invention further relates to a method of attaching a linker arm to multiple organic compounds and a method of attaching a linker arm to a nanocrystal. The present invention further relates to the linker arms herein described and nanocrystals attached to the linker arms herein described. The present invention also relates to nanocrystals and semiconductor nanocrystals in combination with the linker arms of the present invention. The present invention further relates to the attachment of a nanocrystal and a linker arm to an organic compound. The present invention relates to assay systems and assay kits for CNS research, receptor purification, pathogens, environmental contaminants, toxins, and screening for drugs, insecticides, herbicides, and other biologically active substances.

The linker arms and linker arm compound derivatives of the present invention enhance stability and are relatively stable, including stability to biological degradation. The linker arms and the linker arm compound derivatives of the present invention are also advantageous in that they can be synthesized at a relatively low cost.

More specifically, the present invention relates to linker arms such as, for example, carbon-carbon chain linker arms by which biologically active molecules such as CNS drugs and neurotransmitters can be attached to nanocrystals. The attachment of a linker arm of the present invention allows nanocrystals to be used as imaging agents in diverse applications such as biochemical research, CNS research, receptor purification, and high throughput screening for new drugs and other biologically active substances.

Additionally, the present invention relates to linker arms such as, for example, carbon linker arms by which biologically active molecules such as drugs, hormones, etc. can be attached to nanocrystals. The linker arms of the present invention enhance water solubility of nanocrystals and allow nanocrystals to be attached to a diverse range of molecules ranging from drugs to polypeptides and neurotransmitters. The linker arm compounds of the present invention allow nanocrystals to be used as imaging agents in diverse applications such as CNS research, receptor purification, assay systems for pathogens, environmental contaminants, toxins, and a high throughput assay system for new drugs and biologically active molecules.

As stated above, preferably the organic part of the nanocrystal compounds of the present invention are biologically active compounds. Preferably, the biologically active compound is one that will bind to detectable substances, if the substance is present, in the material being analyzed.

In general, any affinity molecule useful in the prior art in combination with a dye molecule to provide specific recognition of a detectable substance will find utility in the formation of the organo-luminescent semi conductor nanocrystal probes of the invention. Such affinity molecules include, by way of example only, such classes of substances as monoclonal and polyclonal antibodies, nucleic acids (both monomeric and oligomeric), proteins, polysaccharides, and small molecules such as sugars, peptides, drugs, and ligands. Lists of such affinity molecules are available in the published literature such as, by way of example, the “Handbook of Fluorescent Probes and Research Chemicals”, (sixth edition) by R. P Haugland, available from Molecular Probes, Inc.

As stated above, the compounds of the present invention enable nanocrystals to be used as probes for neurotransmitters, receptors and transporter proteins. In one embodiment of the present invention, seratonin (5-hydroxytriptamine) is attached to a nanocrystal. Seratonin is a neurotransmitter which has been linked to the regulation of critical behaviors including sleep, appetite, and mood.

The seratonin transporter (SERT) is a 12-transmembrane domain protein responsible for the uptake of seratonin by the cell. The seratonin labeled nanocrystal compounds of the present invention have a measurable ability to block the uptake of tritiated sepatonin by the human and Drosophila seratonin transporter (hSERT and dSERT).

Seratonin labeled nanocrystals (SNACs) of the present invention may be prepared by reacting trioctylphosphineoxide coated nanocrystals with seratonin and tetramethylammonium hydroxide in methanol. The SNACs are isolated by precipitation and purified to remove seratonin. Linkage of the seratonin presumptively occurs through the lone pair of the hydroxyl to the Cd surface atoms of the nanocrystal. hSERT and dSERT are transfected into HeLa cells via a vaccinia virus/T7 expression system. Following expression of the transfected transporters, the cells are assayed for uptake of tritiated seratonin in the presence of increasing concentrations of SNACs. Kᵢ values, the concentration at which half the SNACs are bound to the transporter, are determined by nonlinear regression. The values [Kᵢ(hSERT)=74 uM, Ki(dSERT)=29 uM] indicate SNACs can effectively interact with the seratonin recognition site of the transporter.

These results suggest that highly fluorescent, seratonin labeled nanocrystals can be used as probes for SERT. These probes assist in determining the structure of SERT, including the number of gene products (SERT proteins) that are required to assemble a functional unit, and following transporter movement within the cell.

The present invention enables nanocrystals to be used as imaging agents, which results in an assay system that is superior to traditional immunoassay systems because, among other things, several wavelengths can be used to induce fluorescence. The linker arm can be attached to a number of different ligands, thus enabling them to be used in high throughput screening and receptor purification. The linker arm is stable and not as subject to enzymatic degradation as other linker arms may experience. The linker arm of the present invention also enhances the solubility of the nanocrystal, and can be readily derivitised. This enables a wide range of molecules to be attached to the nanocrystals. The linker arm of the present invention is not as temperature sensitive as many immunoassay systems, and thus is likely to have a longer shelf life. Further, the linker arm of the present invention is also robust and therefore not susceptible to extremes of pH that may denature and degrade peptide linkers.

As stated above, the linker arm of the present invention may have the following formula:

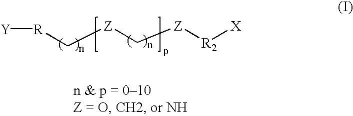

wherein Y represents the attachment point to the nanocrystal and X represents the attachment point of an organic compound. R is a bond or is selected from the group consisting of SH, O(CH₂₍ₙ₎O)ₙSH, NH(CH₂₍ₙ₎O)ₙSH, NH(CH₂₍ₙ₎NH)SH, S(CH₂₍ₙ₎O)ₙSH, and S(CH₂₍ₙ₎S)SH. n is 1-10, with S being attached to the nanocrystal.

R₂ is a bond or selected from the group consisting of carbonyl, NH, S, CONH, COO, S, C₁₋₁₀ alkyl, carbamate, and thiocarbamate.

When n and p are 1 or more, the resulting carbon or carbon chain may be substituted.

Preferably, z is CH₂. Preferably n and p are 1-5.

Furthermore, the linker arm of the present invention may have the following formula:

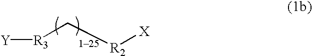

Wherein Y is an attachment point for a nanocrystal, X is an attachment point of an organic compound,

R₂ is a bond or a group selected from the group consisting of:

carbonyl,

NH,

O,

S,

CONH,

COO,

S,

C₁₋₁₀ alkyl,

carbamate, and

thiocarbamate.

R₃ is:

SH;

O(CH₂₍ₙ₎O)ₙSH;

NH(CH₂₍ₙ₎O)ₙSH;

NH(CH₂₍ₙ₎NH)SH;

S(CH₂₍ₙ₎O)ₙSH;

S(CH₂₍ₙ₎S)SH.

n is 1-10, with S being attached to the nanocrystal.

Preferably, n=1 to 5.

The length of the linker arms of the present invention may be increased or shortened in order to increase the solubility of the nanocrystal drug conjugate and increase the affinity of the ligand for its target protein.

The linker arms of the present invention include the following compounds:

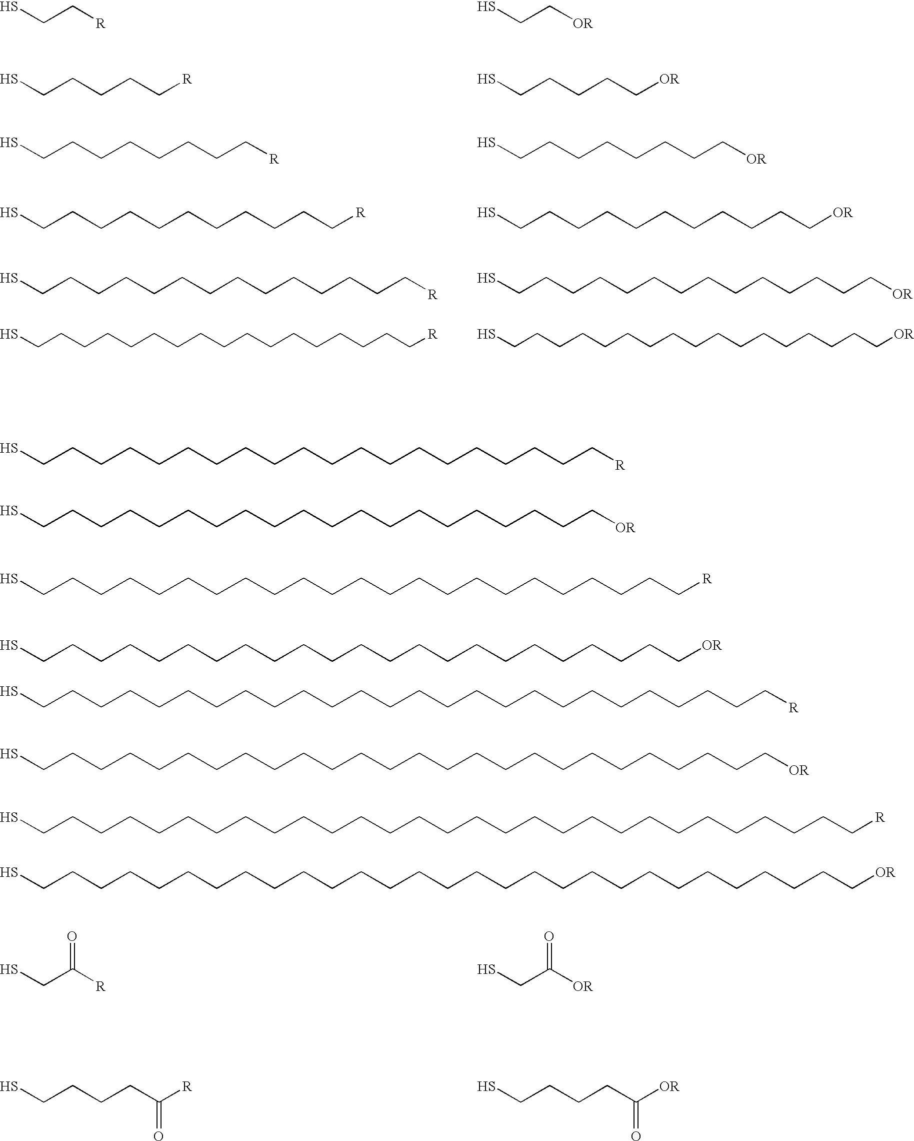

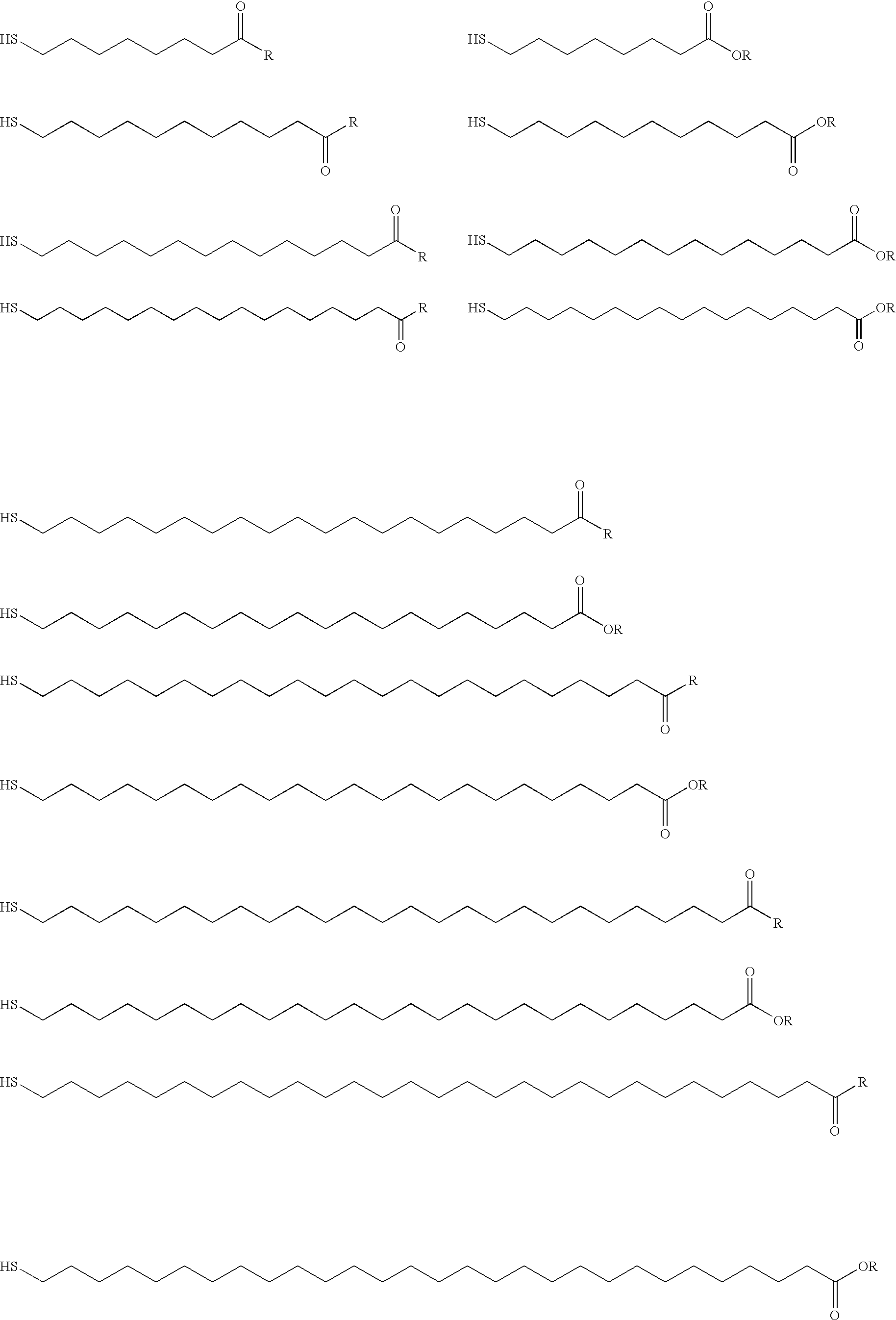

In the above examples, R represents the point of attachment of an organic compound.

The nanocrystal compounds of the present invention include the following, with S being attached to the nanocrystal:

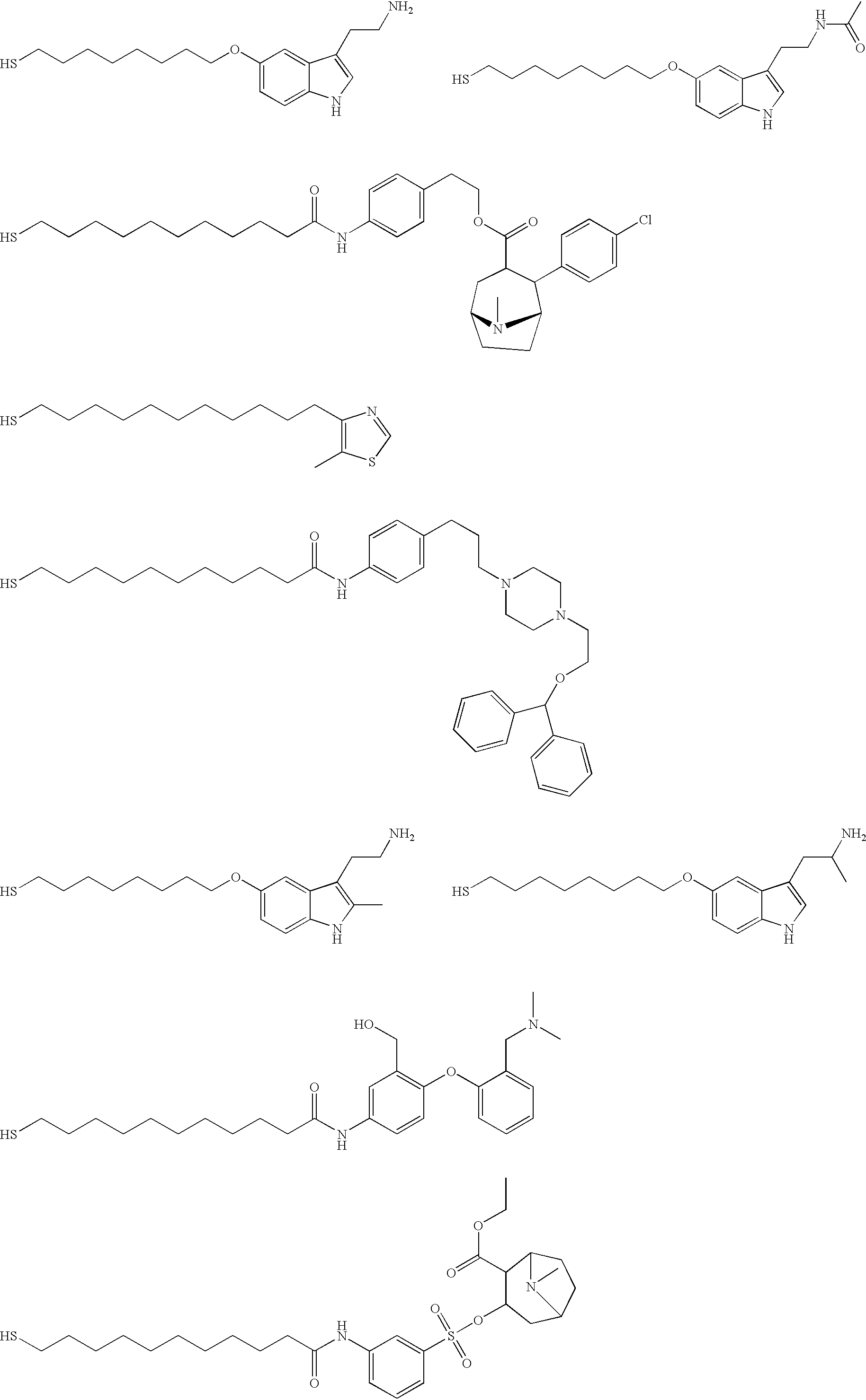

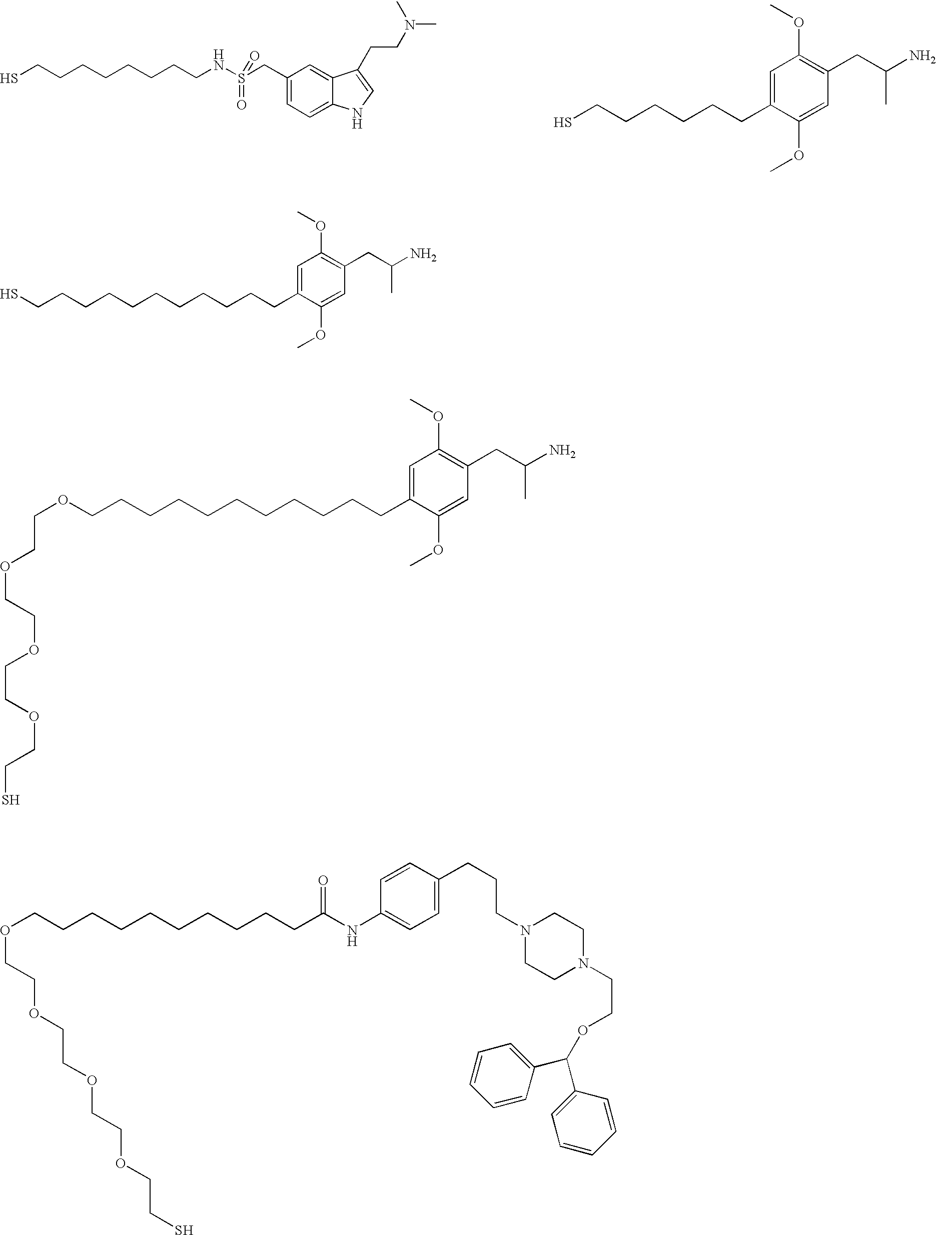

Nanocrystal compounds of the present invention include compounds that comprise of nanocrystals with the following specific and preferred features: a CdSe core, ZnS shell, generally their cores are less than 25 nm, in diameter. The surrounding ZnS shell is typically 10 to 20 nm in thickness, and the ligand coated core shells are water solubilised by the addition of a mercapto acetic acid co-solubility ligand.

By attaching antibodies to nanocrystals via a linker arm of the present invention, nanocrystals can be made to bind to specific antigens. Accordingly, an embodiment of the present invention is an assay kit developed for the detection of a diverse range of substances ranging from environmental contaminant such as DDT, dioxanes, chemical warfare agents, herbicides, pesticides, and pathogenic organisms such as Ecoli 0157 and Salmonela.

For example, the present invention comprises a process for treating a material, such as a biological material, to determine the presence of a detectable substance in the material. The process comprises contacting the material with a nanocrystal conjugated compound of the present invention, washing unbound nanocrystal conjugated compound away, and exposing the material to energy such as an electromagnetic source or particle beam capable of exciting the nanocrystal conjugated compound of the present invention, and causing a detectable fluorescence to occur in the nanocrystal conjugated compound of the present invention. Thus enabling the location and distribution of a particular substance within the biological material to be determined.

The nanocrystal compounds of the present invention may be used in the assays described in U.S. Pat. No. 5,990,479.

One assay system of the present invention is a high throughput fluorescence assay to identify novel ligands that might be effective antidepressants or ligands that might help combat cocaine addiction. In this assay a known agonist or antagonist for the dopamine receptor or transporter is bound to nanocrystals, and incubated with cells that either naturally express or have been engineered to express dopamine receptors or transporters. After incubating for 12 hours excess ligands are removed by washing and unknown compounds are incubated with the cells for a further 12 hours. The cells are washed again with buffer and a fluorescence assay is performed. Any cells that no longer fluoresce have a high affinity ligand bound to them and this ligand may be used as a lead compound for drug discovery. Such an assay system may be carried out in a conventional multiple well format system, such as the 96 well format.

Chart A, below demonstrates another method of the present invention that may be used to detect biologically active analytes. Chart A describes a sandwich assay system. In chart A, in step 1 monoclonal or polyclonal antibodies raised against a specific analyte or groups of analytes are bound to the surface of the plate. In step 2, the analyte is added and binds to the antibody. In step 3, the unbound analyte is washed away and a nanocrystal antibody conjugated using our linker arm of the present invention is added (once again poly or monoclonal antibodies may be used). In step 3, the unbound nanocrystal antibody conjugates are removed by washing, and a fluorescence assay is performed to determine if the analyte is present in the sample being analyzed and its concentration as a sample with a higher concentration will produce a greater fluorescence. Multiple analytes can be screened for using a conventional 96 well plate format.

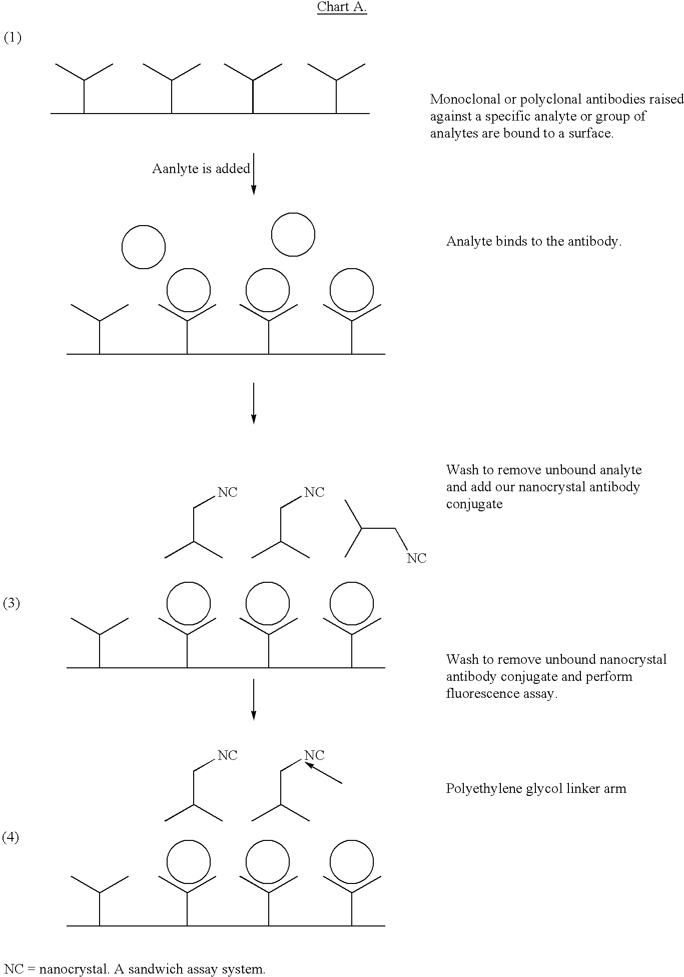

The nanocrystal of the present invention may be used in affinity chromatography, where a compound or biological molecule of interest may be bound to a column. This may then be specifically labeled with the antibody nanocrystal conjugate, substrate nanocrystal conjugate, or drug nanocrystal conjugate of the present invention. The compound could be a drug, a hormone, an enzyme, a protein, a nucleic acid or a receptor. Once the nanocrystal conjugate has bound to the substrate of interest, it may either remain bound to the column or be eluted with the mobile phase. This would enable the isolation and identification of the compound or biological molecule of interest. Unlike fluorescent dyes, nanocrystals are not easily photo-bleached. Therefore, it would be easier to watch the compound or compounds eluting off the column. Also such a system may be applied to several different analytes enabling the identification of several unknowns at once by using different sized nanocrystals conjugated to different ligands. Thus it is theoretically possible to identify different receptor classes or subtypes (e.g. 5-HT receptor subtypes) as they elute off the column. For example it may be possible to differentiate between 5HT2 and 5HT3 receptor subtypes using such a system.

The linker arm acts as a spacer and separates the ligand from the nanocrystal thus possible steric and other interactions between nanocrystals and ligand are minimized. The linker arm may be an ethylene glycol moiety this helps to enhance the solubility in aqueous media. Many affinity chromatographic systems are typically run in such media. The polyether linker arm is also resistant to proteolytic cleavage which may be a problem with other assay systems.

Nanocrystals can be attached to enzymes via linker arms of the present invention. Thus the amino derived carboxylic acid derived poly ethers may be linked to the backbone of the peptide via a peptide bond.

In this instance the linker arm removes the enzyme from the immediate environment of the nanocrystal. This may be important in reducing any effects that the nanocrystal may have upon the enzymes activity. Many such instances could be envisaged particularly if the enzyme or protein undergoes a conformational change during its catalytic cycle (e.g. Hemoglobin). Also the linker arm may increase the catalytic efficiency of the enzyme if the active site or sites are close to the enzymes surface.

Such a system may also be used to identify analytes in a similar manner to the nanocrystal antibody conjugates previously described. It may also be used in high throughput screening where the compounds of interest are bound to wells in plates and the enzyme nanocrystal conjugate is added. An example of this is shown in chart B below.

Compounds A,B,C and D etc are bound to wells on a plate.

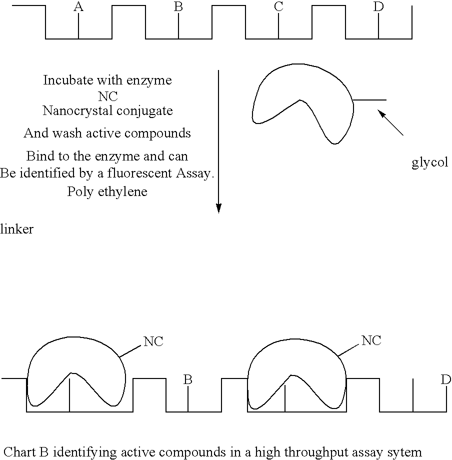

The enzymes substrate or inhibitor may also be bound to the polyethylene glycol nanocrystal conjugate. In this instance, the linker arm of the present invention reduces steric hindrance between nanocrystal and enzyme and it enables the substrate to enter the enzymes catalytic or alosteric site, which may not be possible if the substrate were bound to the surface of the nanocrystal (particularly if the site of interest is deep within the enzyme). An assay system that could use this technique as a tool for identifying new drugs is outlined in chart C, below, where compounds that will compete for the site of interest can be identified. If the nanocrystal is bound to an inhibitor via the linker arm of the present invention it is likely that this assay system could also be used to identify other inhibitors of the enzyme.

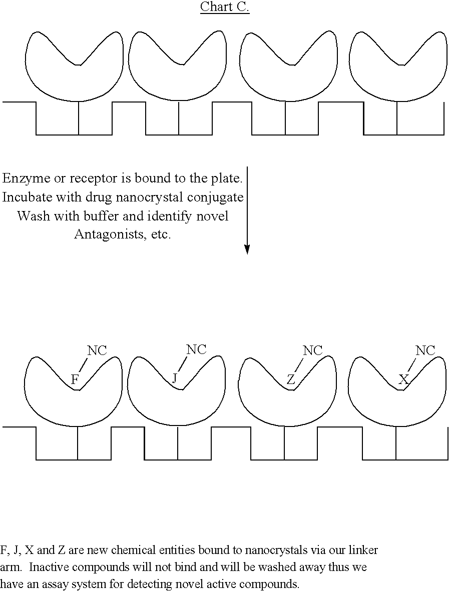

One specific substance may also be bound to the nanocrystal (e.g. a substrate for the enzyme) and a simple competitive assay could be performed with unknown substances in a manner similar to that shown above in chart C. Any substance that has a higher affinity for the site of interest on the enzyme, protein or receptor than the ligand conjugated nanocrystal would displace the ligand conjugated nanocrystal resulting in a loss off fluorescence, thus enabling this system also to be used as a high throughput assay system as well as an analytical tool for environmental contaminants, toxins, and other unknowns.

This system can be applied to receptors rather than enzymes. In this case, the nanocrystal is bound to an agonist, antagonist, or natural ligand for the receptor (e.g. Seratonin). This system could be used as an assay system for receptor agonist or antagonist. It would be of interest in neuropharmacology where receptor location and distribution could be mapped. By attaching different sized nanocrystals to different agonists, antagonists, or ligands it may be feasible to develop multiplexing assay systems, thus enabling the effects of drugs and other neurologically active agents to be monitored in whole cell assay systems. Assaying the location and distribution of many membrane bound receptors and transporter proteins is currently difficult using conventional antibody fluorescent dye systems is difficult due to photo-bleaching and the broader emission spectra of dyes.

Nanocrystals may be attached to DNA or RNA via the linker arm of the present invention. In this case, the major role of the linker arm acts as a spacer and reduces steric hindrance. The DNA or RNA conjugates may be used as a tool in molecular biology for identifying the location and frequency and rate of expression of specific gene sequences. Such a system is outlined in chart D, below.

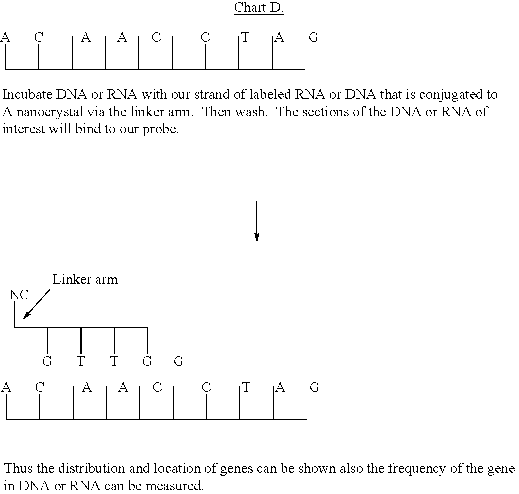

The nanocrystal conjugates of the present invention can also be used in assay systems in the same manner that antibody fluorescent dye conjugates, radio immuno assays, and ELISA are used. Examples of the assay system include routine assays used in medical laboratories such as tests for various disease states, for example HIV, Diabetes, etc.

Other features of the invention will become apparent in the course of the following examples, which are given for illustration of the invention and are not intended to be limiting thereof.

### EXAMPLES

### Example 1

A nanocrystal conjugated biologically active compound of the present invention may be made as follows:

### (±)1-[2,5-Dimethoxy-4-(alkyl)phenyl]-2-aminopropane coated nanocrystals

A linker arm for the above compound may be made as follows:

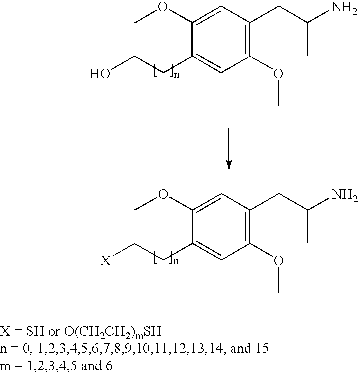

Or alternatively as follows:

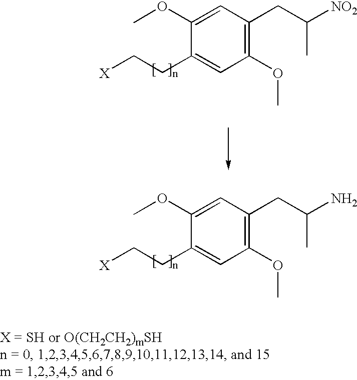

The synthesis of the alcoholic precursor where n=5 is shown in chart 1. The thiol was synthesized by two different routes, these are outlined in charts 2 and 3.

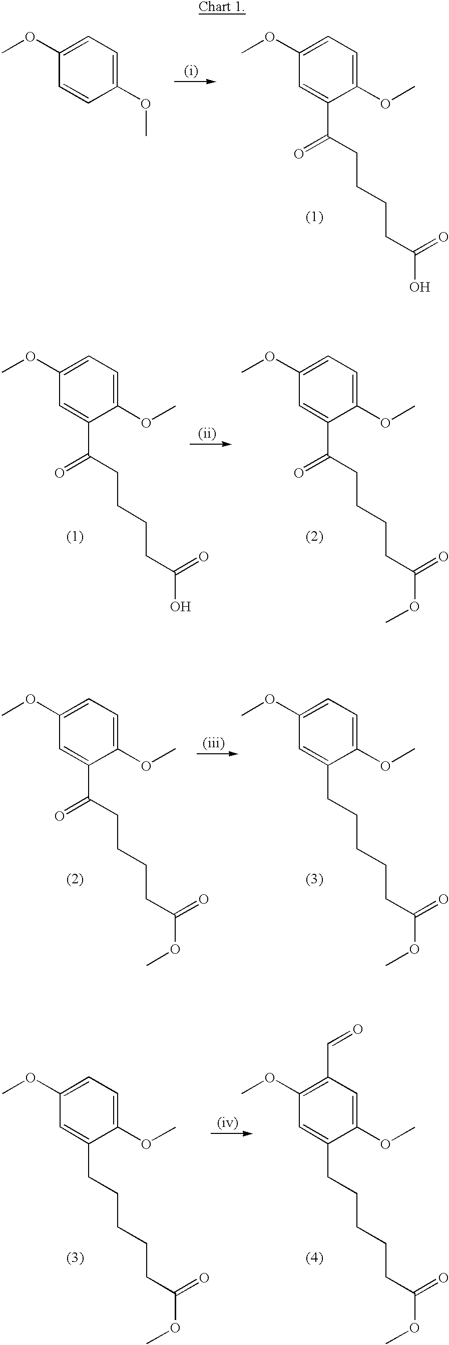

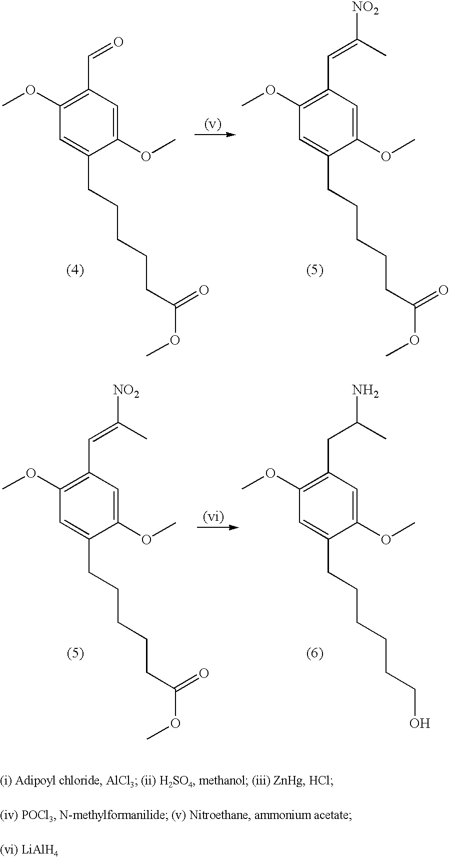

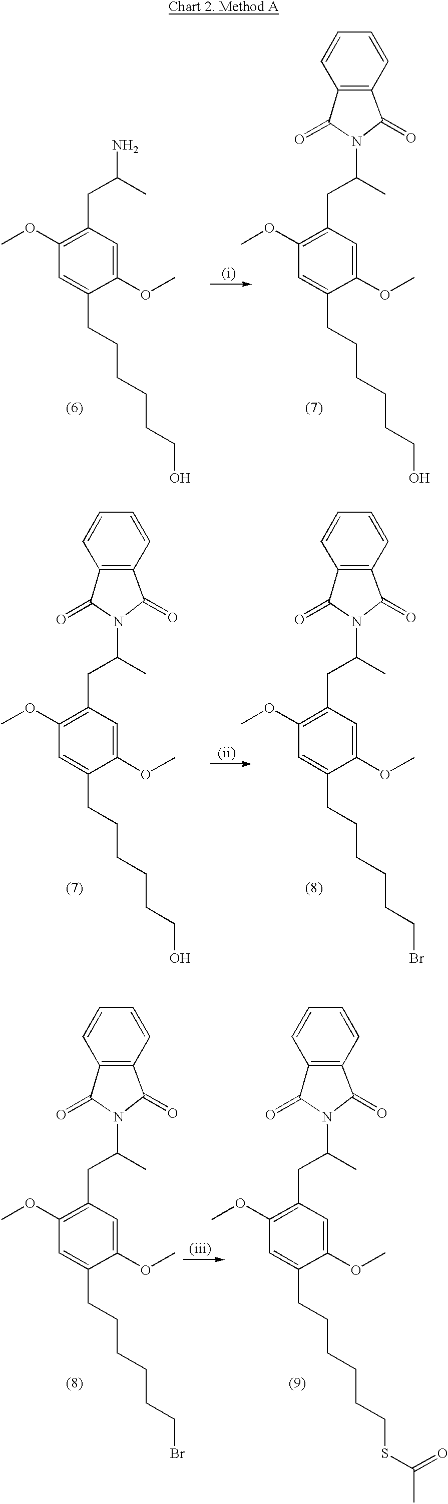

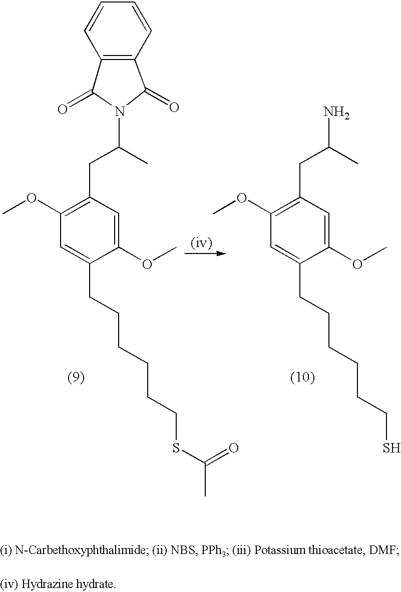

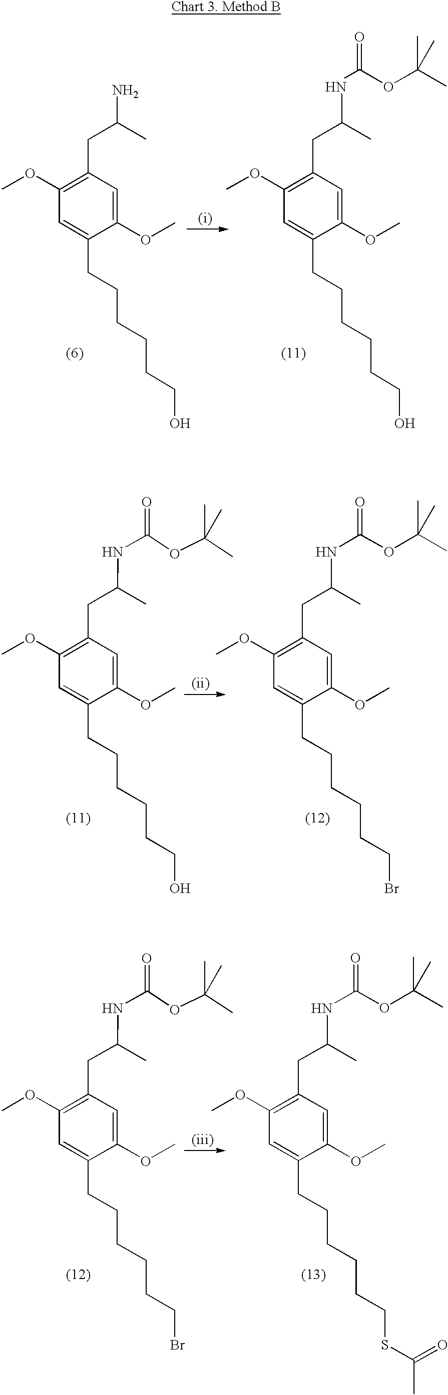

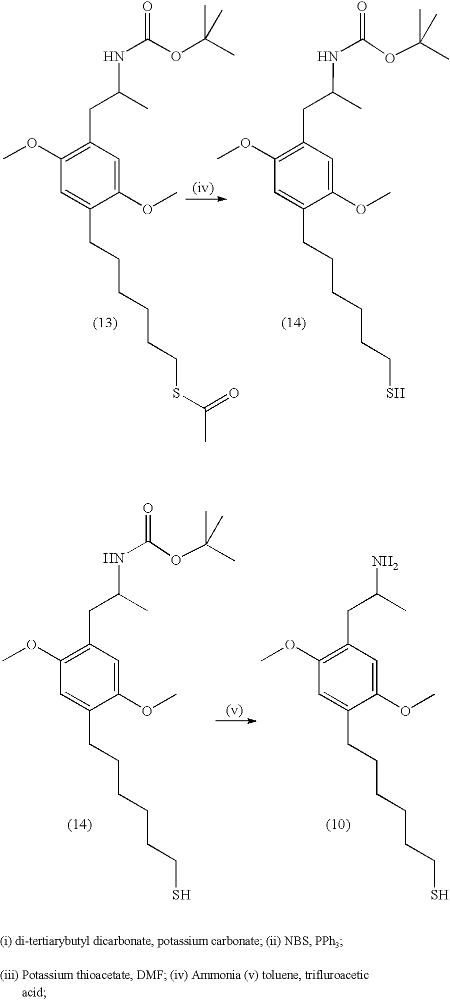

The synthesis of the alkyl thiol where n=11 is outlined in chart 4.

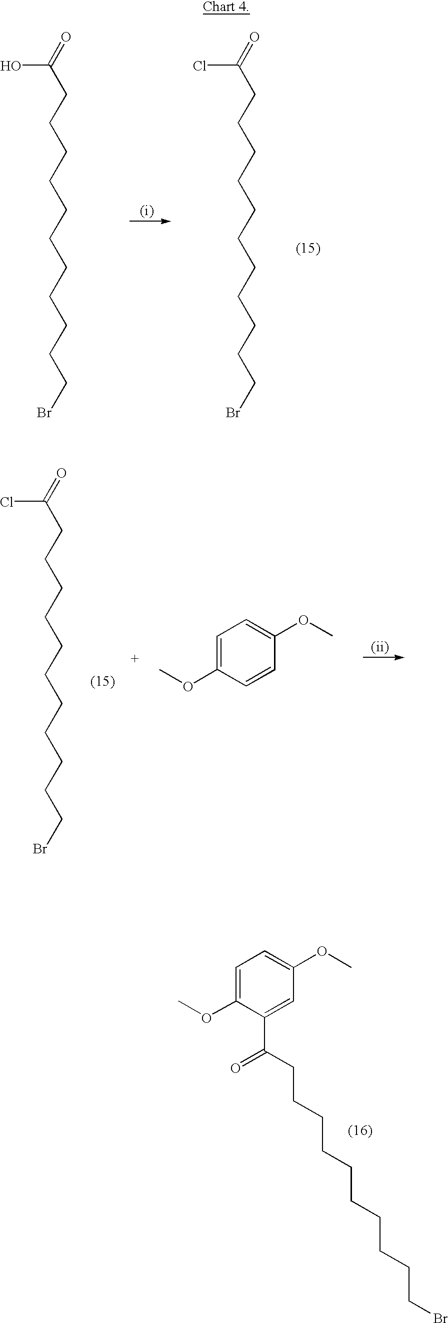

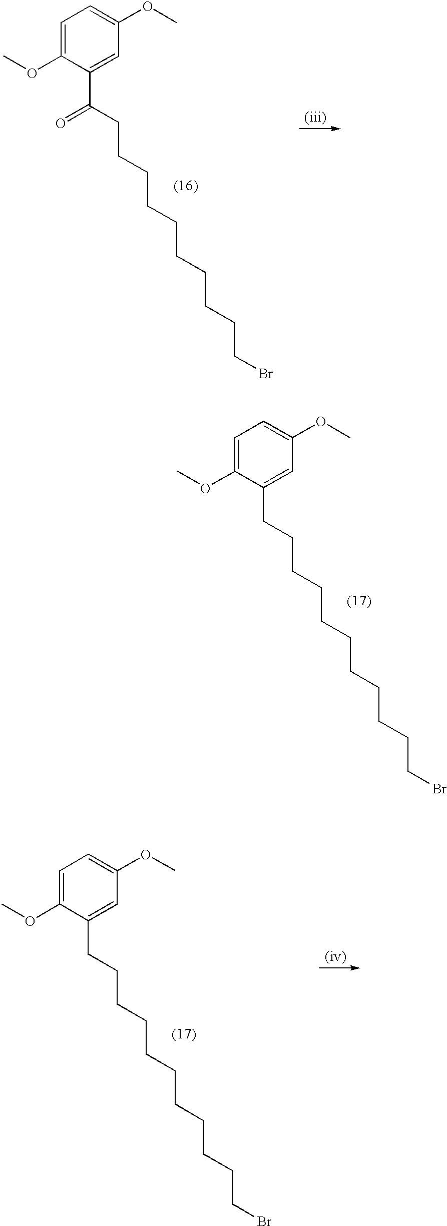

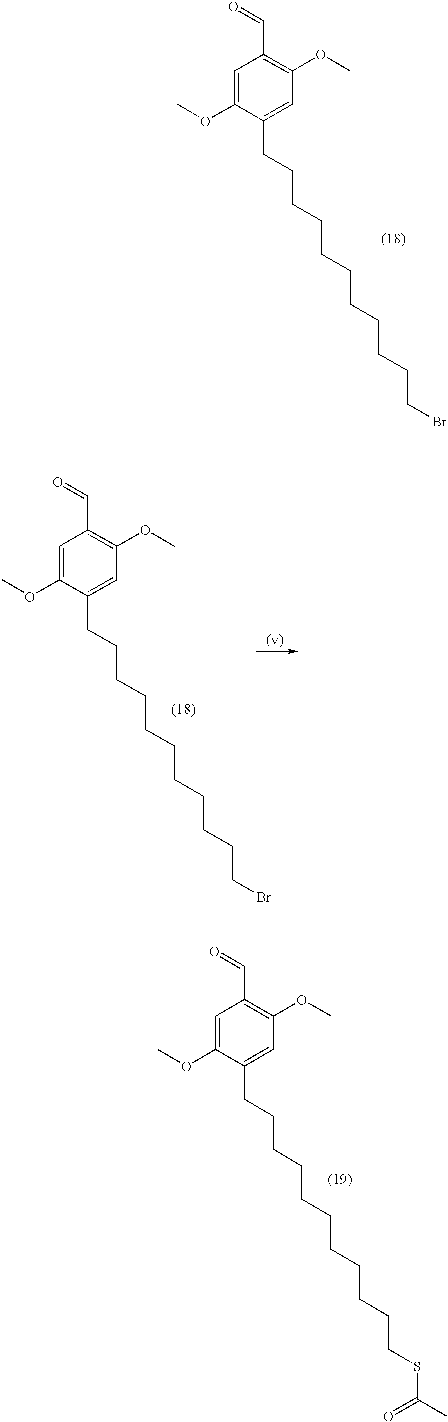

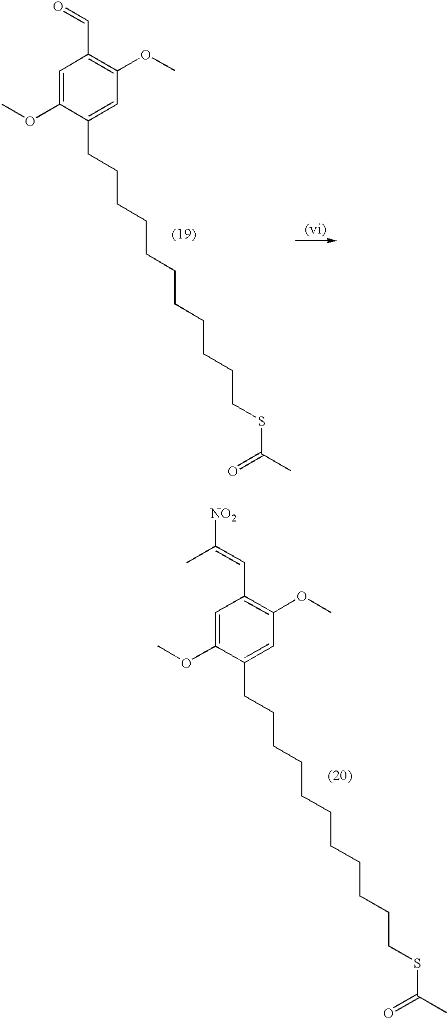

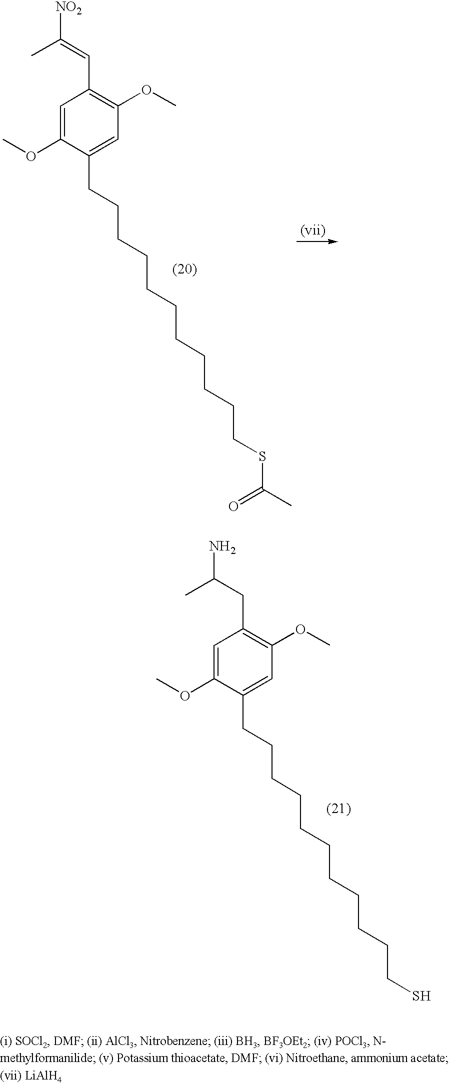

The following compounds correspond with the above-numbered compounds.

### 6-(2,5-dimethoxyphenyl)-6-oxohexanoic acid (1)

Adipoyl chloride (50 ml) and aluminum chloride (10 g, 7.4 mmols) are dissolved in nitrobenzene (50 ml) and cooled to 0° C., in a three necked 250 ml round bottomed flask equipped with a stirrer, thermometer, addition funnel and a calcium chloride drying tube. 1,4-Dimethoxybenzene (10 g, 7.2 mmols) in nitrobenzene (50 ml) is added drop wise over a 3 hour period and the temperature is maintained below 50° C. The resulting mixture is stirred for a further 2 hours at 0° C. Then crushed ice is added and the reaction mixture is allowed to warm to room temperature over an 18 hour period. The solution is filtered and extracted into sodium hydroxide solution (3M, 3×100 ml). The aqueous solution is acidified using hydrochloric acid (4M) to pH 1. The solution is extracted with diethyl ether (3×200 ml) and the combined ethereal extracts are dried over magnesium sulfate. After the solution is filtered it is evaporated and the product is recrystallized from ethyl acetate:hexane. This gives approximately 11.4 g (60%) of the product as a colorless solid mpt=75-77° C.

### Methyl-6-(2,5-dimethoxyphenyl)-6-oxohexanoate (2)

6-(2,5-dimethoxy-phenyl)-6-oxohexanoic acid (4.2 g, 160 mmols) is added to methanol (100 ml) in a 250 ml round bottomed flask equipped with a stirrer and a reflux condenser. A catalytic quantity of concentrated sulfuric acid (2 drops) is added. The solution is heated at reflux over a period of 18 hours with stirring. After cooling to room temperature the solution is evaporated under reduced pressure and the crude product is dissolved in diethyl ether (100 ml). This is washed with sodium carbonate (saturated, 50 ml) and water (50 ml). It is dried over magnesium sulfate filtered and evaporated under reduced pressure. The product is purified using column chromatography on silica gel eluted with dichloromethane. This gives approximately 4.2 g (94%) of the product as a pale yellow oil.

### Methyl-6-(2,5-Dimethoxyphenyl)hexanoate (3)

Powdered zinc (22.5 g) is added to a solution of mercuric chloride (0.94 g) in concentrated hydrochloric acid (0.93 ml) and water (23.1 ml). This suspension is shaken for 5 minutes and the liquid is decanted. The amalgamated zinc is placed in a 500 ml 3 necked flask and concentrated hydrochloric acid (12 ml) is added. The flask is heated to cause a gentle reflux and a solution of Methyl-6-(2,5-dimethoxyphenyl)-6-oxohexanoate (4.2 g, 15 mmols) in methanol (7 ml) and concentrated hydrochloric acid (23 ml) is added drop wise. The mixture is heated at reflux for 3 hours following the addition of (35) then filtered. The aqueous solution is extracted with diethyl ether (4×100 ml) and the combined ethereal extracts are washed with sodium bicarbonate (saturated, 50 ml) and water (50 ml). After drying over magnesium sulphate the solution is filtered and evaporated. The product is purified by column chromatography on silica gel eluted with dichloromethane 98%:methanol. This gives approximately 1.35 g (33%) of the product as a pale yellow oil.

### Methyl-6-(2,5-dimethoxybenz-4-formyl)hexanoate (4)

A mixture of phosphorus oxychloride (1 ml) and N-methylformanilide (1.81 g) are allowed to incubate at room temperature for 30 minutes, in a 25 ml round bottomed flask equipped with a stirrer and a reflux condenser. Methyl-6-(2,5-Dimethoxyphenyl)hexanoate (1 g, 4 mmols) is added and the mixture is heated for 2 hours. After cooling to room temperature water (50 ml) is added and the mixture is left standing at room temperature for 18 hours. Then the solution is extracted with dichloromethane (2×100 ml) dried over magnesium sulphate filtered and evaporated. The resulting oil is leached with boiling hexane's (4×100 ml) and the combined solutions are evaporated under reduced pressure. Purification of the product is accomplished by column chromatography on silica gel eluted with dichloromethane 98%:methanol. This gives approximately 0.4 g (35%) of the product as a colorless solid mpt=74-76° C.

### Methyl-6-(2,5-Dimethoxy-4-(2-nitroprop-2-ene)phenyl)hexanoate (5)

Methyl-6-(2,5-dimethoxybenz-4-aldehyde)hexanoate (1 g, 3.4 mmols) is added to glacial acetic acid (100 ml) in a 200 ml round bottomed flask equipped with a reflux condenser and a stirrer. This is followed by ammonium acetate (0.272 g) and nitro ethane (1 ml). The mixture is heated at reflux for 4 hours and then it is evaporated. The product is purified by column chromatography on silica gel eluted with ethyl acetate 25%:Hexane 75%. This gives approximately 0.42 g (35%) of the product as a yellow solid mpt=57-58° C.

### 1-(2,5-Dimethoxyphenyl-4-(6-hydroxyhexyl))-2-aminopropane (6)

Methyl-6-(2,5-Dimethoxyphenyl-4-(2-nitroprop-2-ene))hexanoate (0.42 g, 1.2 mmols) is dissolved in dry diethyl ether (100 ml) in a 250 ml round bottomed flask equipped with a reflux condenser and a stirrer. A solution of lithium aluminum hydride (1M, 14 ml) is added and the mixture is heated at reflux for 48 hours under nitrogen. It is stirred for a further 2 days under nitrogen at room temperature. The solution is cooled to 0° C. in an ice-acetone bath and sulfuric acid (8%) is added until hydrogen evolution ceased. The aqueous solution is separated and washed with diethyl ether (2×50 ml). Then the aqueous solution is basified with sodium bicarbonate to pH 8 and the aluminum salts were removed by filtration. The inorganic salts are air died and washed with dichloromethane (2×100 ml) and the aqueous solution is extracted with (2×100 ml). The combined organic extracts are dried over magnesium sulfate filtered and evaporated to yield approximately 0.25 g (66%) of the product as a colorless solid.

### 6-(2,5-Dimethoxy-4-(2-[N,N-phtalimido]propyl)phenyl)hexanol (7)

1-(2,5-Dimethoxy-4-(6-hydroxyhexyl))-2-aminopropane (0.25 g, 0.8 mmols) is dissolved in tetrahydrofuran (10 ml) in a 50 ml round bottomed flask equipped with a stirrer. A solution of sodium bicarbonate (0.1 g) in water (10 ml) is added and N-Carbethoxy phalimide (0.175 g, 0.8 mols). The mixture is stirred at room temperature for 18 hours. Then extracted with dichloromethane (2×50 ml). The combined organic extracts are washed with water (20 ml) dried over magnesium sulfate, filtered and evaporated under reduced pressure. The product is purified by column chromatography on silica eluted with ethyl acetate 50%:hexanes. This gives approximately 0.326 g (95%) of the product as a colorless solid mpt=82-83° C.

### 6-(2,5-Dimethoxy)-4-(2-[N,N-phtalimido]propyl)phenyl)hexylbromide (8)

6-(2,5-Dimethoxy-4-(2-[N,N-phtalimido]propyl)phenyl)hexanol (0.2 g,0.4 mmols) is dissolved in dichloromethane (20 ml) and cooled to 0° C. in a 50 ml round bottomed flask equipped with a thermometer, dropping funnel and a stirrer. Triphenyl phosphine (0.13 g, 0.51 mmols) in dichloromethane (10 ml) is added drop wise to the solution of (40). After stirring for 30 minutes a solution containing N-bromosuccinamide (0.09 g, 0.5 mmols) in dichloromethane (10 ml) is added drop wise over 10 minutes. The solution is stirred for 10 minutes at 0° C. after the addition of N-bromosuccinamide is complete. Then it is allowed to warm to 22° C. and it is stirred for 2 hours at this temperature. After which the solvent is removed under reduced pressure and the product is purified by column chromatography on silica eluted with ethyl acetate 50%:hexanes. This gives approximately 0.06 g (27%) of the product as a yellow oil.

### 6-(2,5-Dimethoxy-4-(2-[N,N-phtalimido]propyl)phenyl)hexylthioacetate (9)

6-(2,5-Dimethoxy)-4-(2-[N,N-phtalimido]propyl)phenyl)hexylbromide (0.28 g, 0.57 mmols) is dissolved in dry dimethyl formamide (10 ml), in a 25 ml round bottomed flask equipped with a stirrer and 4 A molecular sieves (10 pellets) are added. The solution is stirred for 1 hour at room temperature, before the addition of potassium thioacetate (0.13 g, 0.00114 mols). Stirring is continued at room temperature for a further 18 hours. After which it is filtered and diethyl ether (100 ml) is added to the solution. The organic solution is washed with water (2×20 ml), hydrochloric acid (1M, 1×20 ml), water (2×20 ml) and sodium bicarbonate (0.1M, 1×20 ml). It is dried over magnesium sulphate filtered and evaporated under reduced pressure. The product is purified by column chromatography on silica eluted with ethyl acetate 33%:hexanes. This gives approximately 0.23 g (82%) of the product as a pale yellow oil.

### 6-(2,5-Dimethoxy-4-(2-aminopropyl)phenyl)hexylthiol (10)

Method A:

6-(2,5-Dimethoxy-4-(2-[N,N-phtalimido]propyl)phenyl)hexylthioacetate (0.23 g, 0.47 mmols) is dissolved in absolute ethanol (50 ml) in a 500 ml round bottomed flask equipped with a stirrer. Hydrazine monohydrate (15 ml) is added to this solution and the mixture is stirred at 22° C. for 90 minutes. Dichloromethane (200 ml) is added and the solution is washed with water (2×100 ml). The organic solution is dried over magnesium sulphate filtered and evaporated. This gives approximately 0.1 g (68%) of the product as a pale yellow oil.

Method B:

6-(2,5-Dimethoxy-4-(2-[N-(tert-butoxycarbonyl)aminopropyl]phenyl)hexyl thiol (0.041 g, 0.097 mmols) is dissolved in dry toluene (10 ml) in a 25 ml round bottomed flask equipped with a stirrer. Trifluoroacetic acid (0.2 ml) is added the mixture is stirred at 22° C. for 1 hour. The solvent is removed under reduced pressure and the resultant tar is dissolved in dichloromethane (20 ml). This is washed with sodium bicarbonate (0.1M, 1×20 ml) and water (2×10 ml). After drying over magnesium sulfate the solution is filtered and evaporated. This gives approximately 0.021 g (70%) of the product as a pale yellow oil.

### 6-(2,5-Dimethoxy-4-(2-[N-(tert-butoxycarbonyl)aminopropyl]phenyl)hexanol (11)

1-(2,5-Dimethoxy-4-(6-hydroxyhexyl))-2-aminopropane (0.025 g, 0.085 mmols) is dissolved in methanolic hydrochloric acid (30 ml) and this is evaporated. Once all the methanol has been removed the resulting solid is dissolved in water (10 ml) and potassium carbonate (0.25 g) is added all at once followed by tertiary butyl carbonic anhydride (0.2 g, 0.0011 mols). The mixture is stirred at room temperature overnight and then extracted with dichloromethane (3×50 ml). The combined organic extracts are dried over magnesium sulphate filtered and evaporated. The product is purified by column chromatography on silica eluted with dichloromethane 95%:methanol. This gives 0.018 g (47%) of the product as a colorless solid.

### 6-(2,5-Dimethoxy-4-(2-[N-(tert-butoxycarbonyl)aminopropyl]phenyl)hexylbromide (12)

6-(2,5-Dimethoxy-4-(2-[N-(tert-butoxycarbonyl)aminopropyl]phenyl)hexanol (0.018 g, 0.045 mmols) is dissolved in dichloromethane (20 ml) and cooled to 0° C. in a 50 ml round bottomed flask equipped with a stirrer. Triphenyl phosphine (0.13 g, 0.049 mmols) in dichloromethane (10 ml) is added drop wise followed by N-bromosuccinamide (0.09 g, 0.05 mmols) in dichloromethane (10 ml). The solution is stirred at 0° C. for 5 minutes following the addition of N-bromosuccinamide and then the solution is allowed to warm to 22° C. It is stirred at 22° C. for 2 hours after which the dichloromethane is removed under reduced pressure and the product is purified using column chromatography on silica eluted with ethyl acetate 50%:hexanes. This gives approximately 0.07 g (30%) of the product as a yellow oil.

### 6-(2,5-Dimethoxy-4-(2-[N-(tert-butoxycarbonyl)aminopropyl]phenyl)hexylthioacetamide (13)

6-(2,5-Dimethoxy-4-(2-[N-(tert-butoxycarbonyl)aminopropyl]phenyl)hexylbromide (0.07 g, 0.015 mmols) is dissolved in dry dimethylformamide (2 ml) in a 25 ml round bottomed flask equipped with a stirrer, 4 A molecular sieves (6 pellets) are added and the mixture is stirred at 22° C. for 1 hour. After which potassium thioacetate (0.035 g, 0.03 mmols) is added. The solution is stirred for 18 hours at 22° C., filtered and diethyl ether (50 ml) is added. This is washed with hydrochloric acid (0.1M, 1×10 ml), water (2×10 ml), sodium bicarbonate (0.1M, 1×10 ml) and water (1×10 ml). The organic solution is dried over magnesium sulfate filtered and evaporated. Then the product is purified using column chromatography on silica eluted with ethyl acetate 50%:hexanes. This gives approximately 0.051 g (73%) of the product as a yellow oil.

### 6-(2,5-Dimethoxy-4-(2-[N-(tert-butoxycarbonyl)aminopropyl]phenyl)hexyl thiol (14)

6-(2,5-Dimethoxy-4-(2-[N-(tert-butoxycarbonyl)aminopropyl]phenyl)hexylthioacetamide (0.051 g, 0.011 mmols) is dissolved in methanol (5 ml) in a 10 ml round bottomed flask equipped with a stirrer. Methanolic ammonia (25 ml) is added and the mixture is stirred at 22° C. for 3 hours and evaporated. This gives a yellow tar which is dissolved in dichloromethane (50 ml), the organic solution is washed with water (1×20 ml) and dried over magnesium sulfate. After filtering it is evaporated and purified using silica gel column chromatography eluted with ethyl acetate 50%:hexanes. This gives approximately 0.04 g (88%) of 47 as a pale yellow oil.

### 11-Bromoundecanoyl chloride (15)

11-Bromoundecanoyl chloride is synthesised as described by Goodman et. al. In the Journal of medicinal chemistry p390, 1984. A solution of 11-bromoundecanoic acid (10.6 g, 0.04 mols) and thionyl chloride (4 ml 0.07 mols) in DMF (0.5 ml) is stirred at 80° C. for 1 hour. The solution is cooled to room temperature and used in the next step without purification.

### 11-Bromo-1-(2,5-Dimethoxyphenyl)-undean-1-one (16)

A cooled solution of 11-bromoundecanoyl chloride (11.35 g, 0.04 mols) in dry nitrobenzene (20 ml) was added to a solution of 1,4-dimethoxybenezene (29.36 g, 0.21 mols) in dry nitro benzene (60 ml). The solution is cooled to 0° C. and aluminium chloride (8 g, 0.045 mols) is added portion wise over a 1 hour period. The solution is stirred at 0° C. for a further 4 hours. Crushed ice is then added and the solution is extracted into dithyl ether. The etherial solution is dried over magnesium sulfate, filtered and evaporated. The product is purified by dry flash chromatography on silica eluted with ethylacetate/hexanes 50:50, followed by recrystalisation from petroleum spirit. This gives approximately 8 g (50%) as a colorless solid.

### 1-(11-bromoundecyl)-2,5-dimethoxybenzene (17)

Dry tetrahydrofuran (100 ml) is added to 11-Bromo-1-(2,5-Dimethoxyphenyl)-undean-1-one (3 g, 0.008 mols). Borane dissolved in THF (1M, 20 ml, 0.02 mols) and borontrifluoride etherate (1 ml) are added and the mixture is heated at 75° C. for 48 hours. The reaction mixture is then cooled and water (100 ml) is added. The solution is extracted with diethyl ether (3×100 ml) dried over magnesium sulfate and evaporated. This gives approximately 2.97 g (100%) of the product as a colorless oil.

### 1-(11-bromoundecyl)-4-formyl-2,5-dimethoxybenzene (18)

Phosphorous oxychloride (2 ml) and N-methylformanilide (3.62 g) are incubated at room temperature for 30 minutes. 1-(11-bromoundecyl)-2,5-dimethoxybenzene (2.97 g, 0.008 mols) is added and the mixture is stirred at 80° C. for 3 hours it is cooled to room temperature and added to crushed ice the resulting mixture is extracted with dichloromethane (2×50 ml) and the combined organic solution is washed with water (2×100 ml). It is dried over magnesium sulfate filtered and evaporated. The product is purified by column chromatography on silica eluted with ethyl acetate/hexanes 50:50. This gives approximately 2.8 g (88%) of the product as a brown solid.

### 11-(4-formyl-2,5-Dimethoxy-phenyl)undecanylthioacetate (19)

1-(11-bromoundecyl)-4-formyl-2,5-dimethoxybenzene (2.8 g, 0.007 mols) is dissolved in dry dimethylformamide (5 ml) and molecularseives (0.1 g) 4 Å pellets are added followed by potassium thioacetate (0.9 g, 0.0079 mols). The mixture is stirred under an inert atmosphere of dry nitrogen for 24 hours. Then diethyl ether (50 ml) is added. The solution is filtered and washed with water (3×100 ml). It is dried over magnesium sulfate filtered and evaporated. The product is purified by column chromatography on silica eluted with ethylacetate/hexanes 30%:70%. This gives approximately 2.6 g (94%) of the product as a brown oil.

### 11-(4-(2-Nitro-prop-2-ene)-2,5-dimethoxy-phenyl)undecanylthioacetate (20)

11-(4-formyl-2,5-Dimethoxy-phenyl)undecanylthioacetate (2.6 g, 0.066 mols) is dissolved in glacial acetic acid (50 ml). Nitroethane (1.9 ml) and ammonium acetate (0.53 g) are added and the mixture is heated at reflux for 6 hours. The solution is cooled to room temperature and water (100 ml) is added. The solution is extracted with dichloromethane (2×100 ml) dried over magnesium sulfate filtered and evaporated. The product is purified by column chromatography on silica eluted with ethyl acetate/hexanes 30%:70%. This gives approximately 1.48 g (50%) of the product as a red oil.

### 11-(4-(2-Amino-propane)-2,5-dimethoxy-phenyl)undecanylthiol (21)

11-(4-(2-Nitro-prop-2-ene)-2,5-dimethoxy-phenyl)undecanylthioacetate (1.5 g, 0.0033 mols) is dissolved in dry diethyl ether (100 ml and lithium aluminium chloride (1 g) is added. The mixture is heated at reflux for 18 hours under a nitrogen atmosphere. Then the reaction mixture is cooled to 0° C. and sulfuric acid (1M, 200 ml) is added. The etherial layer is removed and the aqueous solution is washed with diethyl ether (2×100 ml). The acidic solution is neutralised with base and the salts are removed by filtration. The solids are extracted with dichloromethane (2×100 ml) and the aqueous solution is extracted with dichloromethane (2×100 ml). The combined organic extracts are dried over magnesium sulfate filtered and evaporated. This yield approximately 0.4 g (35%) of the product as a brown oil.

### Example 2

This example deals with adjusting the length of the arm. The linker arm of the present invention may be derivatized and further lengthened by adding a polyethylene glycol an illustrative example is outlined in

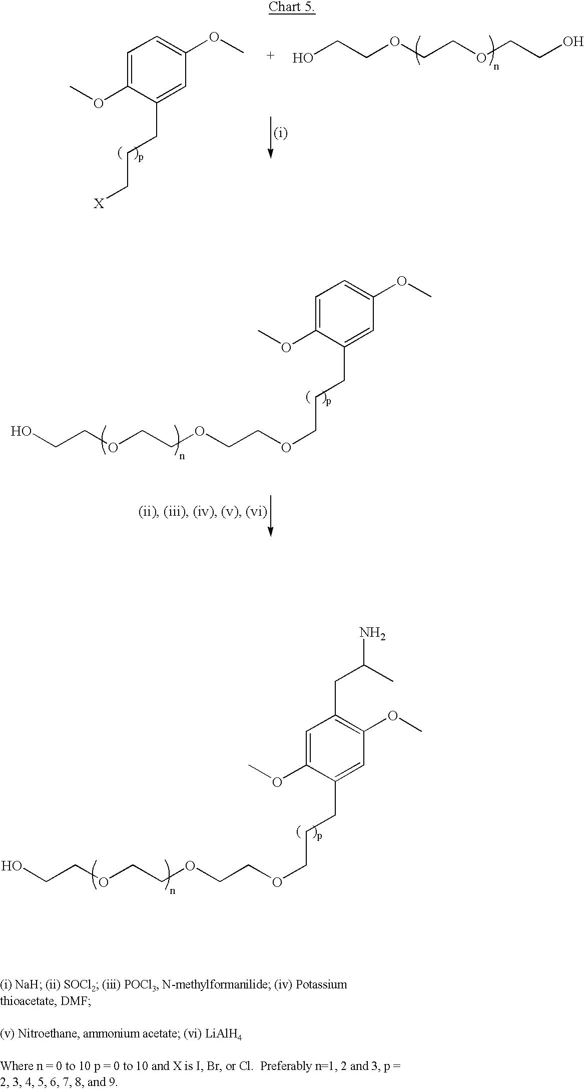

### Example 3

Further example of preparing a nanocrystal conjugated biologically active compound of the present invention. Mercapto-alkyl carboxylic acid (4-{3-[4-(2-benzhydryloxy-ethyl)-piperazin-1-yl]-propyl}-phenyl)-amide conjugated nanocrystals

The linker arm used in the ligand (II), above, is made as follows:

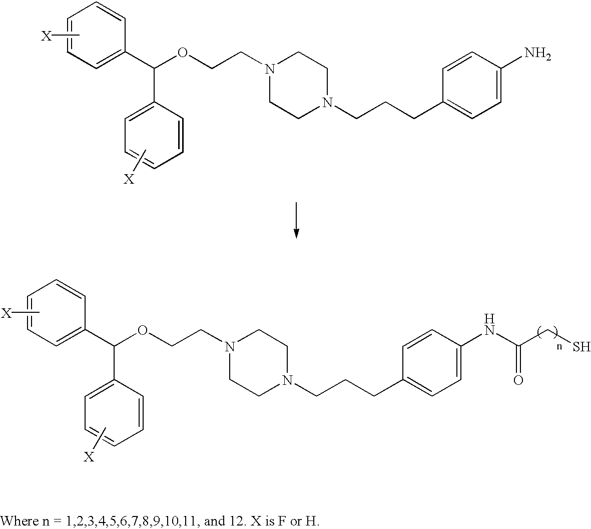

The synthesis of the alkyl amide where n=10 is outlined in chart 6.

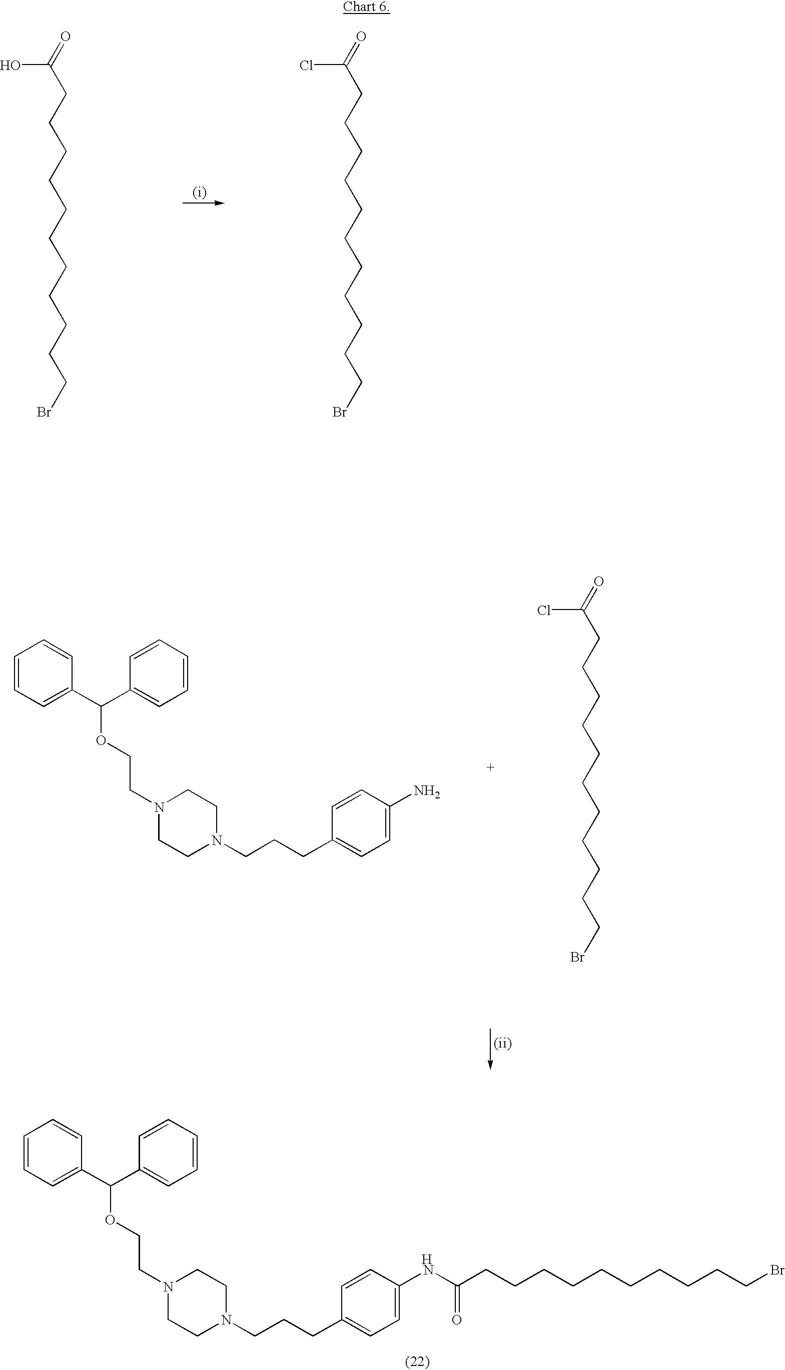

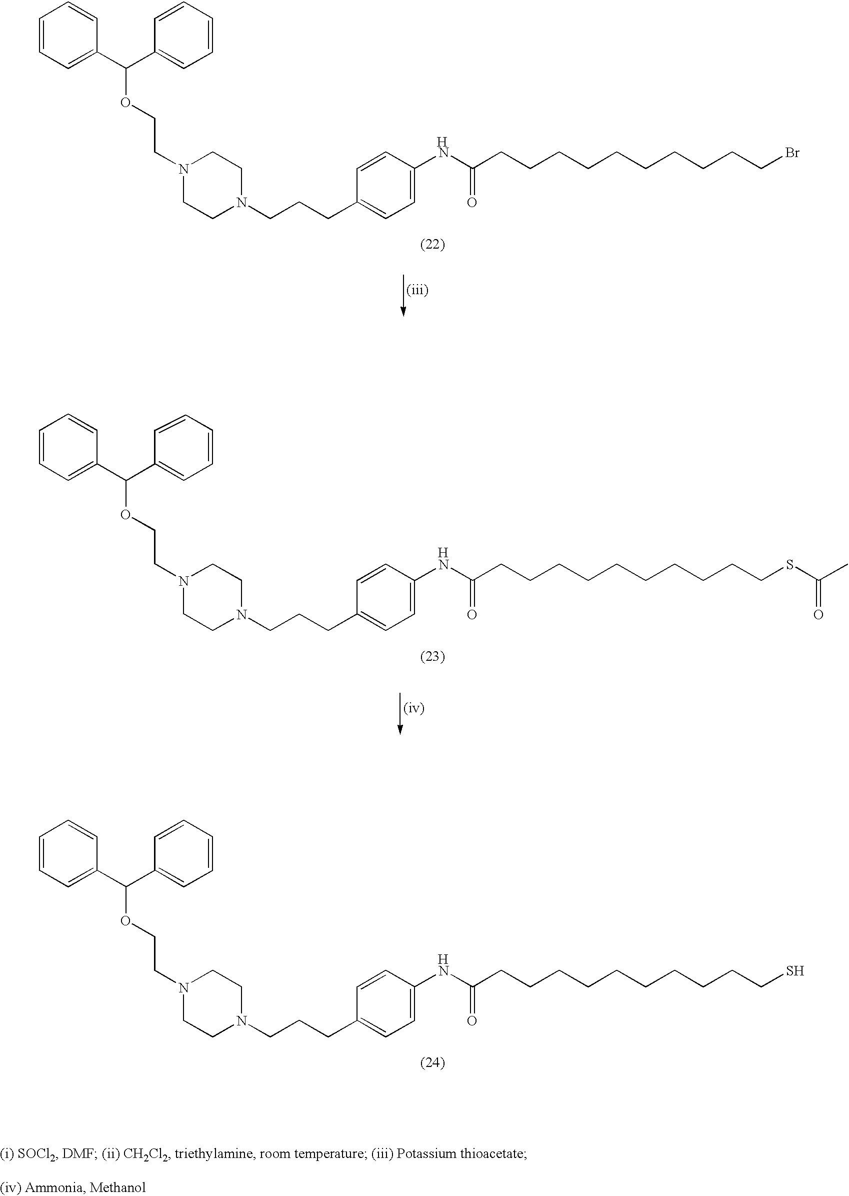

### 11-Bromo-Undecanoic acid (4-{3-[4-(2-benzhydryloxy-ethyl)-piperazine-1-yl]-propyl}-phenyl)-amide (22)

11-bromoundecanoic acid (0.42 g, 0.0016 mols) is dissolved in dry dichloromethane (50 ml) and thionyl chloride (1 ml) is added. A catalytic quantity of dry dimethyl formamide (1 drop) is added and the mixture is heated at reflux for 30 minutes. The solvent is removed under reduced pressure and the acid chloride is dissolved in dry dichloromethane (20 ml) This solution is added dropwise to a methylene chloride solution containing 1-[2-[bisphenylmethoxy]ethyl]-4-(3-(4-aminophenyl)propyl)piperazine (0.64 g ,0.00 mols) and triethylamine (1 ml). The solution is stirred at room temperature for 4 days. Then the solvent is removed under reduced pressure and the product is purified on a silica column eluted with a gradient system running from dichloromethane to dichloromethane:methanol (5%). This gives approximately 0.19 g (23%) of the product as a pale yellow oil.

### Thioacetic acid S-[10-(4-{3-[4-(2-benzhydryloxy-ethyl)-piperazin-1-yl}-propyl}-phenylcarbamoyl)-decyl]ester (23)

11-Bromo-Undecanoic acid (4-{3-[4-(2-benzhydryloxy-ethyl)-piperazine-1-yl]-propyl}-phenyl)-amide (0.19 g, 0.00035 mols) is dissolved in dry dimethyl formamide (4 ml) and potassium thioacetate (0.08 g, 0.0007 mols) is added. The mixture is stirred under nitrogen for 48 hours and then it is diluted with diethyl ether (100 ml). This is filtered and evaporated under reduced pressure. The product is purified by column chromatography on silica gel eluted with a gradient system running from dichloromethane to dichloromethane:methanol 5%. This gives approximately 0.058 g (31%) of the product as a pale yellow oil.

### 11-Mercapto-undecanoic acid (4-{3-[4-(2-benzhydryloxy-ethyl)-piperazin-1-yl]-propyl}-phenyl)-amide (24)

Thioacetic acid S-[10-(4-{3-[4-(2-benzhydryloxy-ethyl)-piperazin-1-yl}-propyl}-phenylcarbamoyl)-decyl]ester (0.058 g, 0.00011 mols) is dissolved in methanol (10 ml) and methanolic ammonia (10 ml) is added. The mixture is stirred at room temperature for 18 hours and evaporated. The product is purified by column chromatography on silica gel eluted with a gradient system running from dichloromethane to dichloromethane:methanol 7%:triethylamine 3%. The base is obtained as a yellow oil and this is converted to the oxalate salt by precipitation from methanol. This gives approximately 0.030 g (51%) of the product as a white solid.

### Example 4

This example demonstrated how the linker arm of the present invention may be derivatized and lengthened. The linker arm of the present invention may be derivatized and further lengthened by adding a polyethylene glycol an illustrative example is outlined in chart 7.

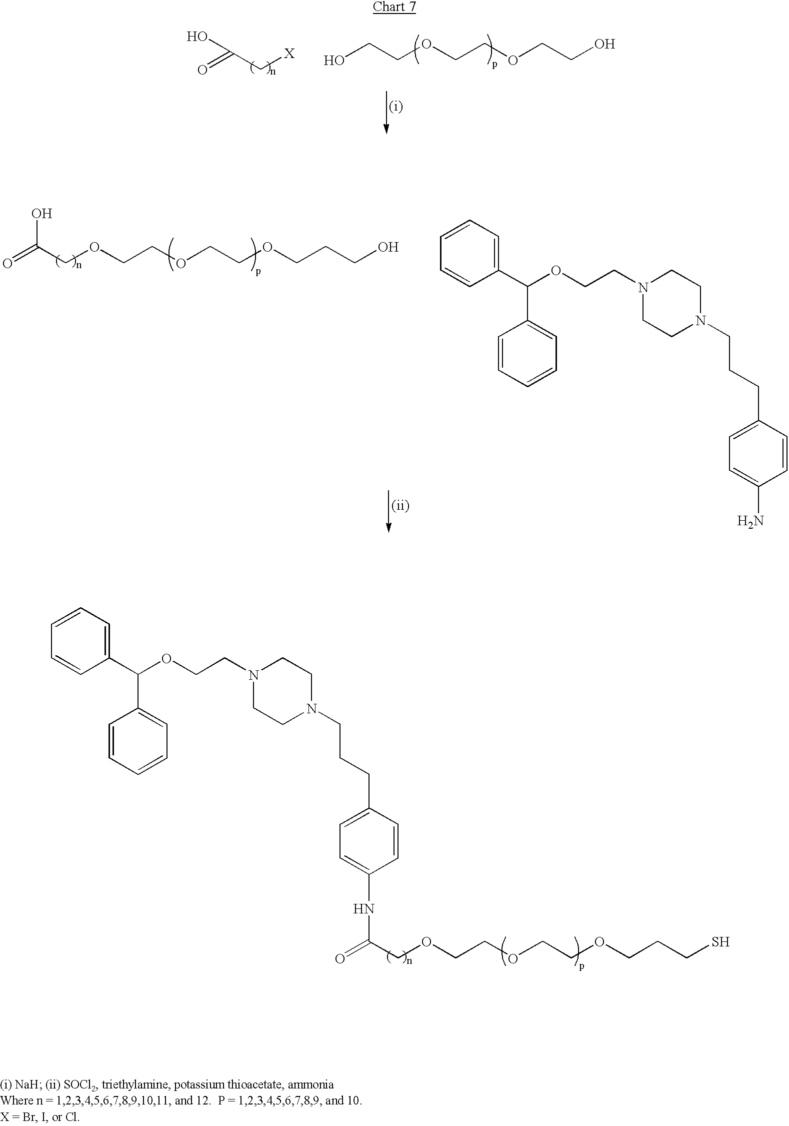

### Example 5

Attachment of biologically active compounds to the linker arm A biologically active organic compound may be attached to the linker arm as

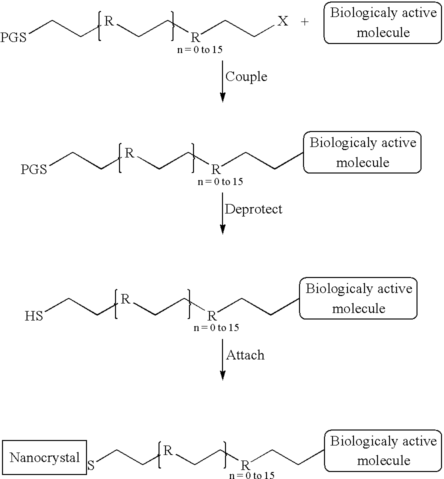

follows:

Where X is Cl, Br, I, OTs, OMs, OTf, NH₂, SH, OH, C═O, COCl, CO₂H, etc. The biologically active molecule is attached to the linker arm via a functional group or a methylene group. R may be O, NH, S, CH₂, etc. PG is a protecting group and may be para-methoxy benzyl, benzyl, a thioamide, a thio ether, etc.

### Example 6

### Attaching linker arms to nanocrystal core shells

This example discloses a method of attaching linker arms of the present invention to nanocrystal core shells. An example of the methodology used is outlined below:

9 mg of trioctylphosphine oxide coated core shells are weighed out and suspended in pyridine (2 ml). The concentration and thus the number of moles of nanocrystals may be determined before hand using UV-vis spectroscopy. This suspension is stirred at 60° C. for 24 hours, N-(4-(3-[4-(2-Benhydryloxyethyl)piperazine-1-yl]propyl)phenyl-2-[2-(2-mercaptoetoxy)ethoxy]acetamide (25), (100 mg) is dissolved in dichloromethane (100 ml) and 2.7 ml of this solution is added to the solution of nanocrystals. This gives approximately 100 ligands per core shell. The solution is stirred at 60° C. under argon for 2 hours. Upon cooling to room temperature the solution is added to hexanes. Ligand coated core shells crystallise out of solution and are collected by filtration.

The water solubility of the ligand functionalised core shells may be increased if necessary by using a modification of the method of Fred Mikulec (private communication). Mercaptoacetic acid (1 ml) and dimethyl formamide (1 ml) are added to the ligand coated core shells and stirred at room temperature under argon for 2 hours. After cooling to room temperature the solution is diluted with dimethyl formamide (100 ml) and potassium teriary butoxide (1.61 g) is added. The resulting solid is collected by centrifugation and is washed with tetrahydrofuran (4×100 ml) and methanol (7×100 ml). The product is collected by centrifugation to yield 45 mg of 1-[2-bisphenylmethoxy]ethyl]-4-(3-(4-(3,6-dioxa-8-thiol)octanamidophenyl)propyl piperazine (25) coated nanocrystals. After drying the precipitate under reduced pressure for 4 days at room temperature the ligand coated cores can be dissolved in a minimum quantity of buffer in a pH range of 6 to 8.

This invention thus being described, it will be obvious that the same may be varied in many ways. Such variations are not to be regarded as a departure from the spirit and scope of the present invention, and all such modifications as would be obvious to one of ordinary skill in the art are intended to be included within the scope of the following claims.

All cited patents and publications referred to in this application are herein expressly incorporated by reference.

We claim:

1. A nanocrystal linker arm of the following formula: 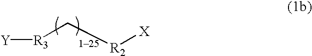
wherein Y is an attachment point for a nanocrystal, X is an attachment point for an organic compound,
R₂ is a bond or a group selected from the group consisting of:
carbonyl,
NH,
O,
S,
CONH,
COO,
S,
C₁₋₁₀ alkyl,
carbamate, and
thiocarbamate.
R₃ is:
SH;
O(CH₂₍ₙ₎O)ₙSH;
NH(CH₂₍ₙ₎O)ₙSH;
NH(CH₂₍ₙ₎NH)SH;
S(CH₂₍ₙ₎O)ₙSH;
S(CH₂₍ₙ₎S)SH.
n is 1-10, with S being attached to the nanocrystal.

2. The linker arm of claim 1, wherein the attachment point for an organic compound is for an biologically active compound.

3. The linker arm of claim 1, wherein the attachment point is for organic compounds selected from the group consisting of: seratonin or seratonin derivatives, cocaine analogues, phenyl tropane analogues, phenylisopropylamine derivatives, dopamine derivatives, melatonin derivatives, chlormethiazole derivatives, derivatives of RTI-4229-75, and derivatives of GBR 12935.

4. The linker arm of claim 1, wherein Y is an attachment point for nanocrystals with cross sections less than about 200 angstroms.

5. The linker arm of claim 1, wherein Y is an attachment point for nanocrystals selected from the group consisting of CdSe, CdS, PbSe, PbS, and CdTe nanocrystals.

6. The linker arm of claim 1, wherein the linker arm is selected from the group consisting of: 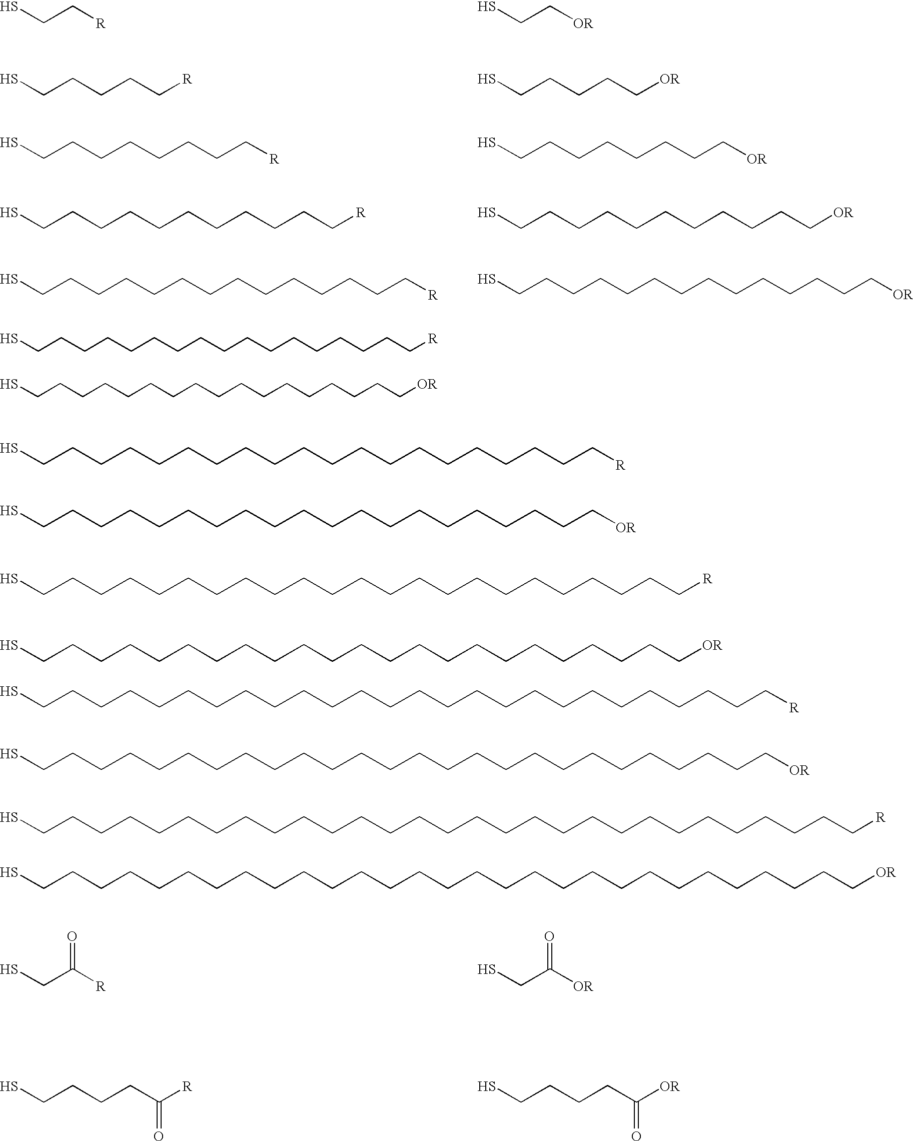 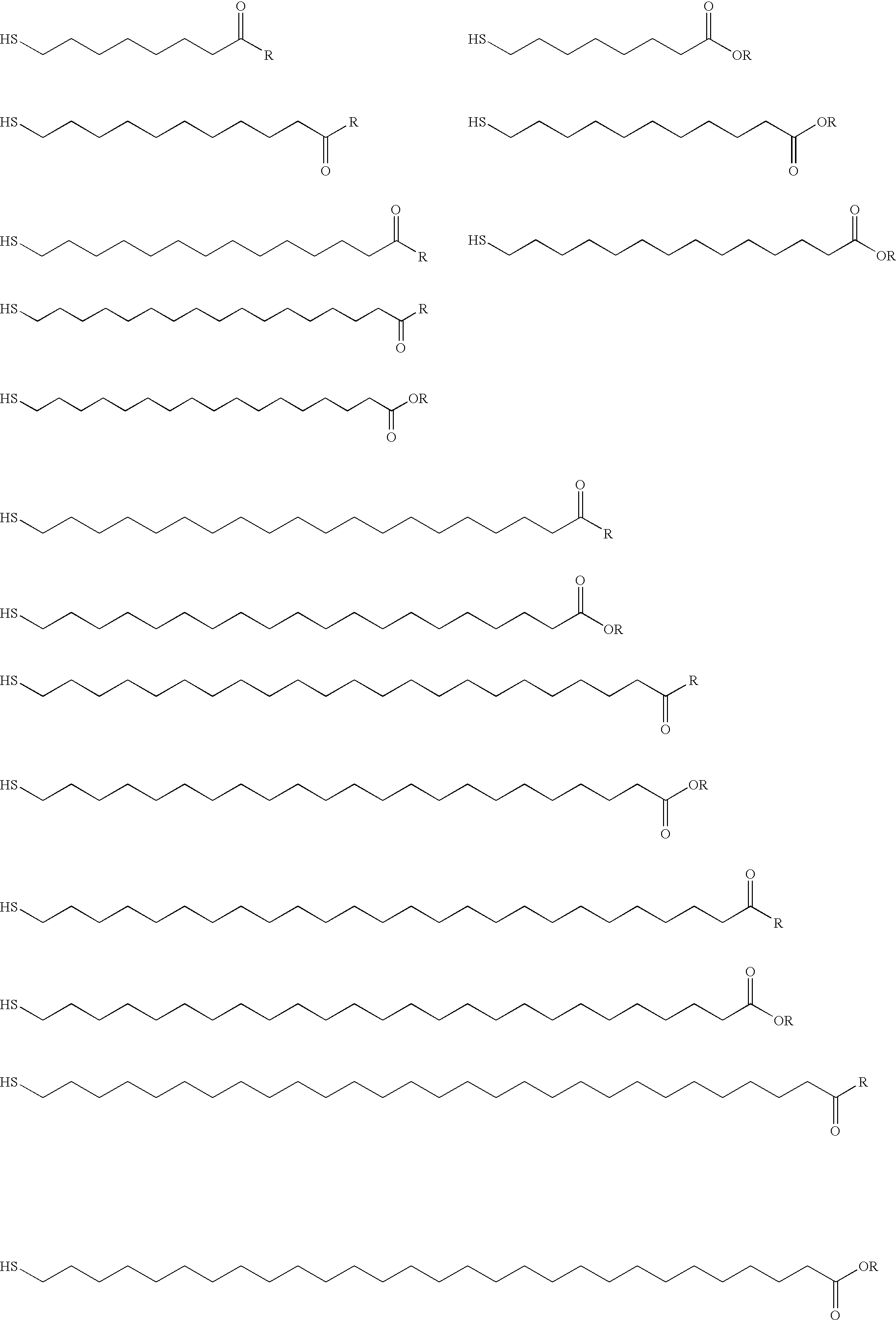
wherein R represents the point of attachment of an organic compound.

7. A nanocrystal compound of the following formula: 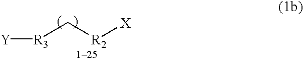
wherein Y is a nanocrystal, X is an organic compound;
R₂ is a bond or selected from the group consisting of:
carbonyl,
O,
NH,
S,
CONH,
COO,
S,
C₁₋₁₀ alkyl,
carbamate, and
thiocarbamate;
R₃ is selected from the group consisting of:
SH,
O(CH₂₍ₙ₎O)ₙSH,
NH(CH₂₍ₙ₎O)ₙSH,
NH(CH₂₍ₙ₎NH)SH,
S(CH₂₍ₙ₎O)ₙSH,
S(CH₂₍ₙ₎S)SH, and
a polyether chain; and
n is 1-10.

8. The nanocrystal compound of claim 7, wherein the organic compound is selected from the group consisting of: seratonin or seratonin derivatives, cocaine analogues, phenyl tropane analogues, phenylisopropylamine derivatives, dopamine derivatives, melatonin derivatives, chlormethiazole derivatives, derivatives of RTI-4229-75, and derivatives of GBR 12935.

9. The nanocrystal compound of claim 7, wherein the organic compound is selected from the group consisting of: 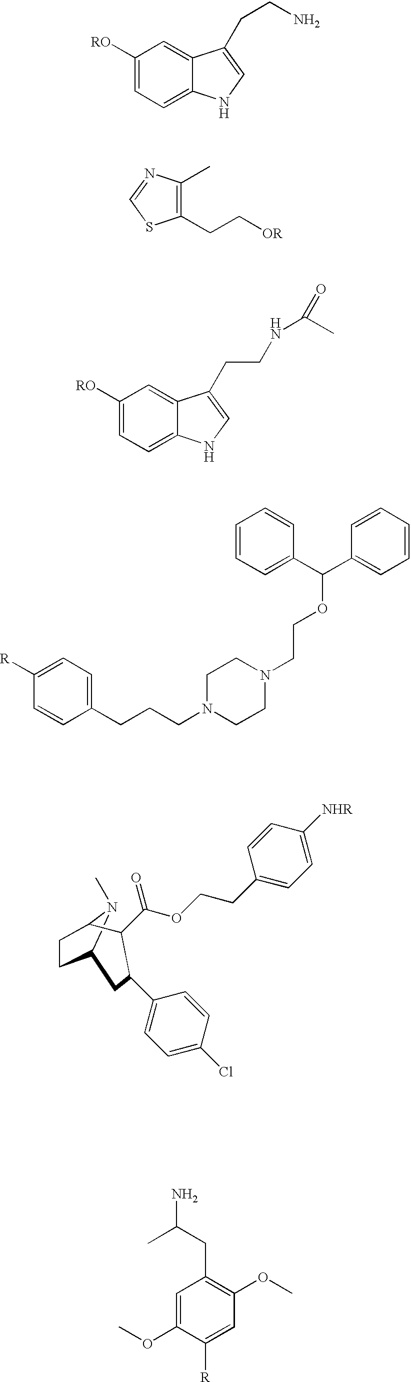
wherein R represents the attachment point to X.

10. The nanocrystal compound of claim 7, selected from the group consisting of: 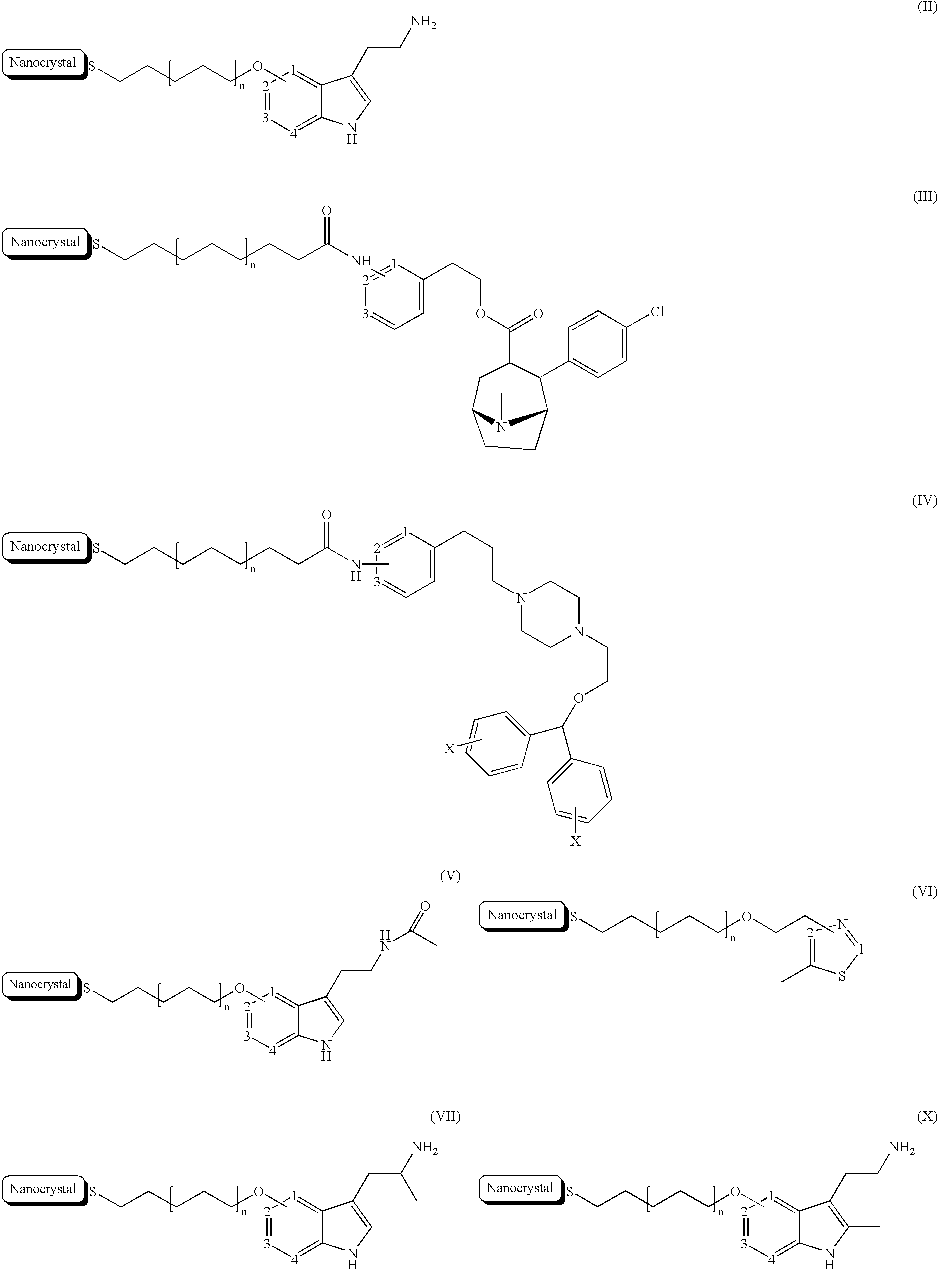 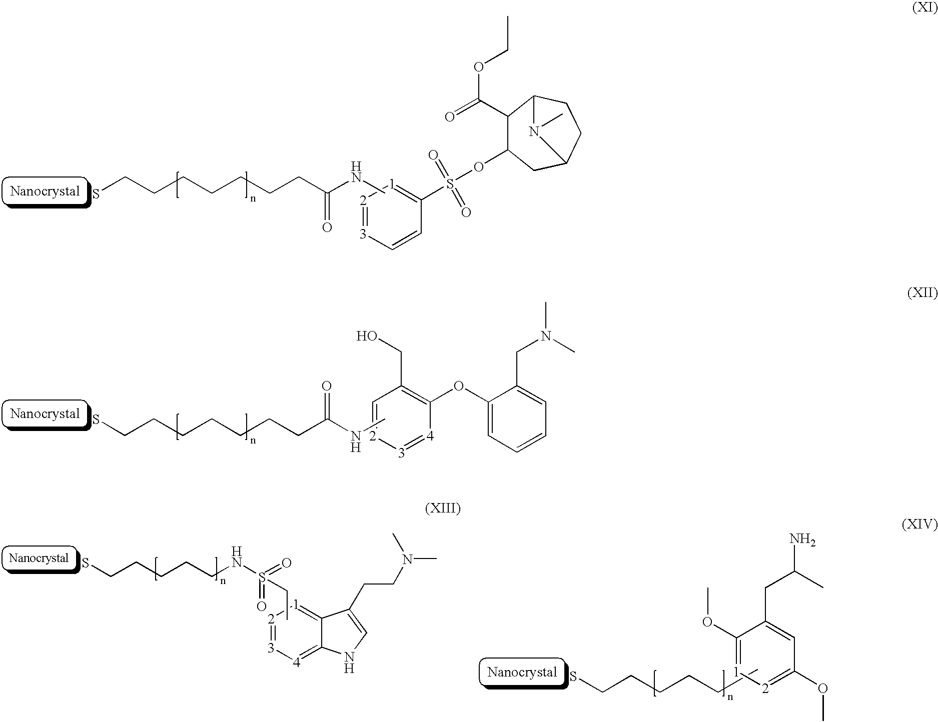
wherein n is 0 to 10 and X is H or halogen.

11. The compound of claim 7, wherein the nanocrystal has a cross section of less than about 200 angstroms.

12. The compound of claim 7, wherein the nanocrystal is selected from the group consisting of CdSe, CdS, PbSe, PbS, and CdTe.

13. The compound of claim 7, wherein the organic compound is capable of binding to an affinity molecule, the affinity molecule being a monoclonal antibody, polyclonal antibody, monomeric nucleic acid, oligomeric nucleic acid, protein, polysaccharide, sugar, peptide, drug, ligand.

14. The compound of claim 7, wherein the organic compound is seratonin.

15. The compound of claim 7, selected from the group consisting of: 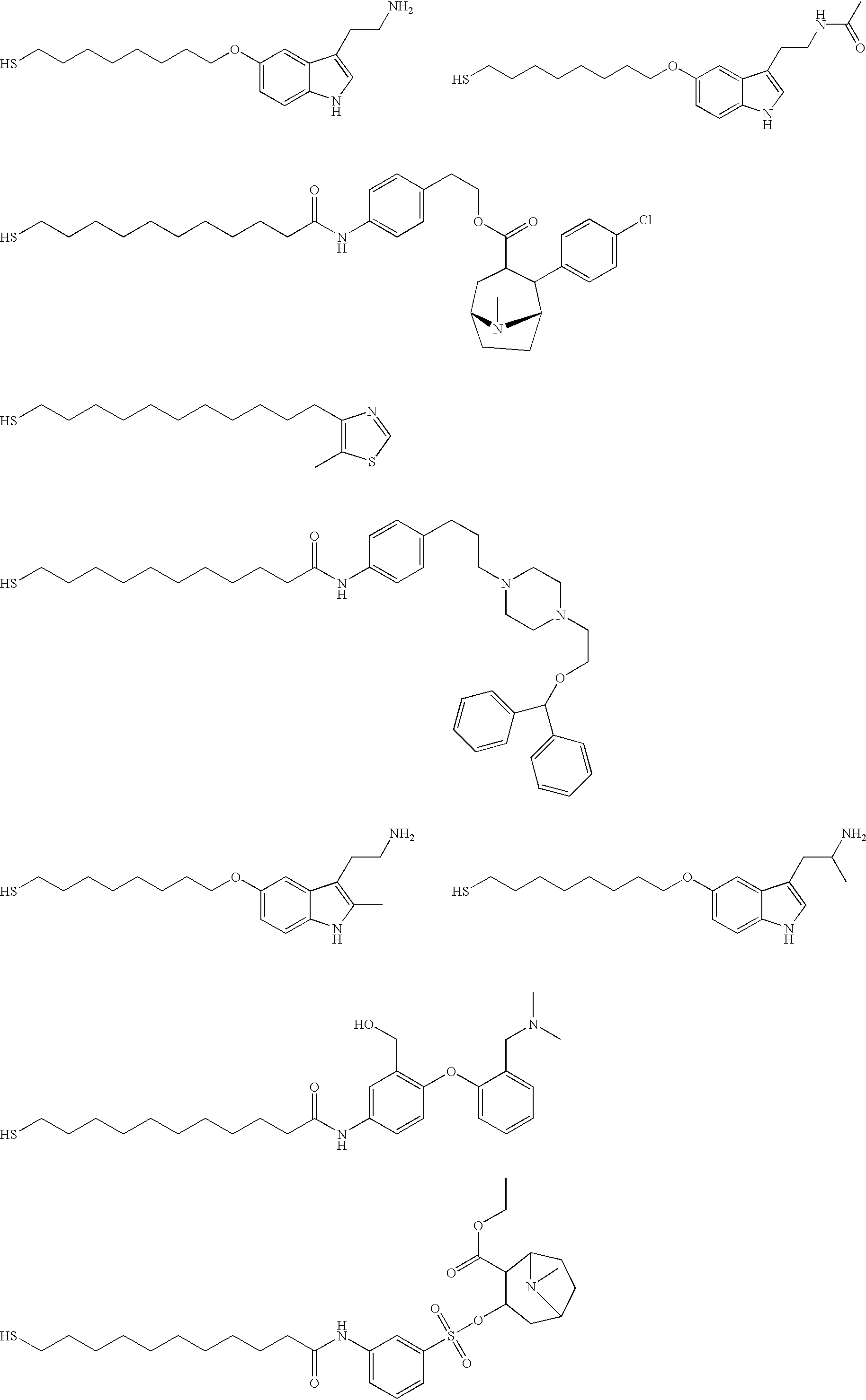 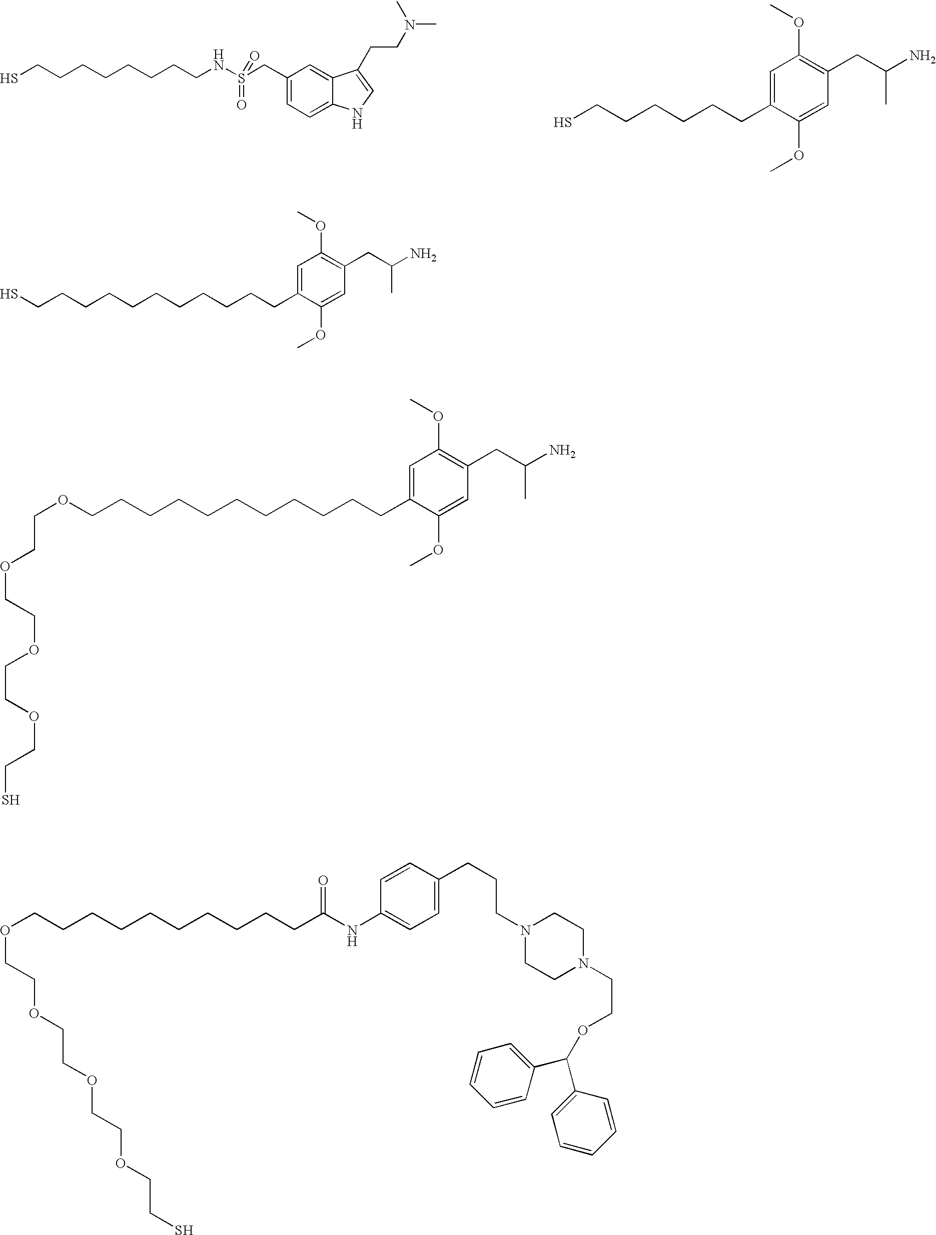
wherein the nanocrystal is attached to the S.

16. The nanocrystal compound of claim 7, wherein the nanocrystal compound is of the following formula:

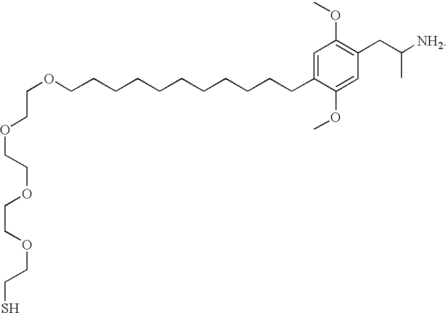

17. The nanocrystal compound of claim 7, wherein the nanocrystal compound is of the following formula:

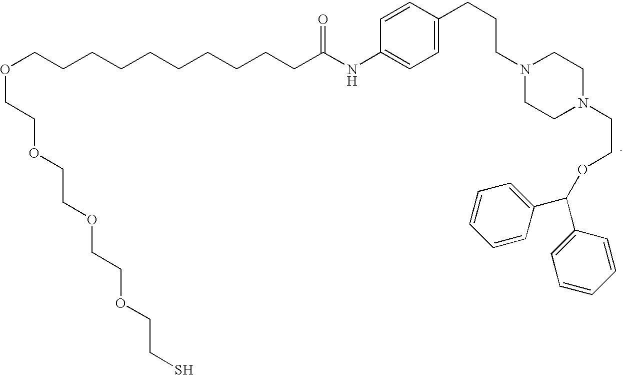

18. The nanocrystal compound of claim 7, wherein the nanocrystal compound is of the following formula:

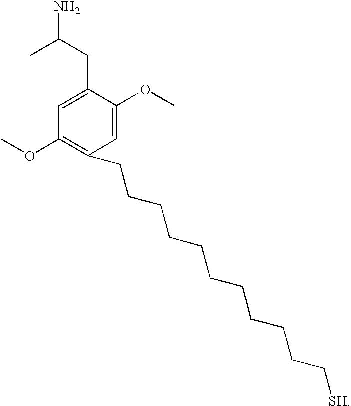

19. The nanocrystal compound of claim 7, wherein the nanocrystal compound is of the following formula:

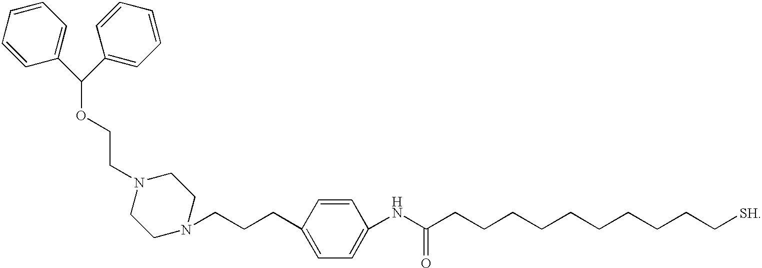

20. A compound of the following formula:

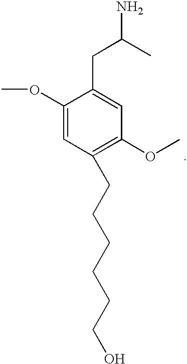

21. A compound of the following formula:

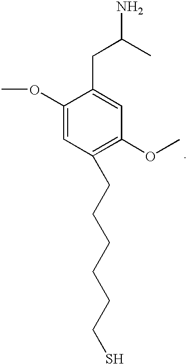

22. A compound of the following formula:

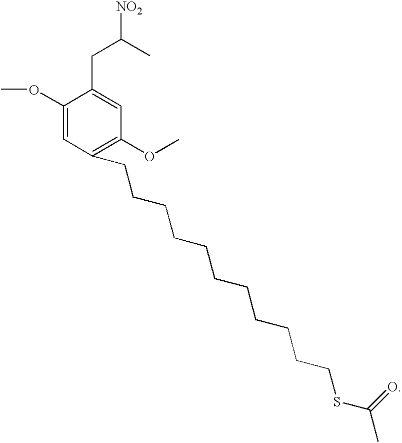

23. A compound of the following formula:

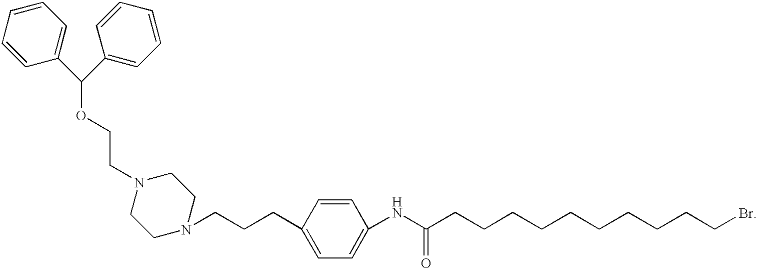

24. A compound of the following formula: [chemical structure]
